(19) United States
(12) Patent Application Publication
(10) Pub. No.: US 2021/0032335 A1
(43) Pub. Date: Feb. 4, 2021

(54) POLYPEPTIDES AND METHODS

(71) Applicant: AUTOLUS LIMITED, London (GB)

(72) Inventors: Martin Pulé, London (GB); Shaun Cordoba, London (GB); Simon Thomas, London (GB); Shimobi Onuoha, London (GB); Daniela Achkova, London (GB); Callum McKenzie, London (GB); James Sillibourne, London (GB)

(21) Appl. No.: 16/967,678

(22) PCT Filed: Feb. 1, 2019
(86) PCT No.: PCT/GB2019/050286

§ 371 Date: Aug. 5, 2020

(30) Foreign Application Priority Data
Feb. 6, 2018 (GB) 1801920.8

(51) Int. Cl. C07K 16/28; C07K 14/725

(52) U.S. Cl. CPC C07K 16/2803; C07K 14/7051; C07K 2319/02; C07K 2317/622; C07K 2319/33; C07K 2319/03

(57) ABSTRACT

The invention relates to a polypeptide comprising (i) an antigen binding domain (ii) a pre-T-alpha domain ectodomain; and (iii) a trans-membrane domain. The invention also relates to nucleic acids, kits, cells, methods and uses.

### BACKGROUND TO THE INVENTION

Chimeric antigen receptors (CARS) are membrane bound proteins which combine an antigen binding domain (e.g. recognising/binding a tumour antigen) and a signalling element for T-cell activation as an artificial type I transmembrane protein. Typically, a CAR is an artificial type I transmembrane protein in the configuration of binding domain-spacer-TM domain-signalling domain. The CAR functions independently of any other receptor molecule. The antigen binding domain is often based on or derived from immunorecognition molecules such as the antigen binding portion of an antibody. “First generation” CARS typically relied on the intracellular domain from CD3Z (also known as CD3 zeta or CD247) for signal transduction. “Second generation” CARS added additional signalling domains from various costimulatory proteins such as CD28, 41BB, or ICOS to the cytoplasmic tail of the CAR to enhance signalling into the T-cell. “Third generation” CARS combined multiple signalling domains such as (CD3Z-CD28-41BB) or (CD3Z-CD28-OX40) in order to increase proliferation and/or increase survival and thereby improve the system.

The most common design of a CAR is a monolithic structure with scFv-spacer-TM-endodomain. Standard monolithic CARs are deficient compared with TCRs for two main reasons: (1) fewer activation domains; (2) lack of physiological control of expression after activation (whereas naturally occurring CD3/TCR complex is under a carefully controlled transcriptional programme which allows rest phase before subsequent activation).

In contrast to the native TCR, these common CAR designs have two main limitations: (1) only a small number of ITAM motifs are activated in contrast to the many from the CD3 complex of a native TCR; (2) Physiological fluctuations of native TCR expression protect T-cells from exhaustion; CAR expression does not fluctuate physiologically without elaborate strategies of integrating the CAR transgene into the TCR locus.

In the early days of CAR development, CARs were developed where the antigen-binding domain replaced the variable region of the TCRalpha/beta. This harnesses the entire CD3 complex for activation. The CD3/TCR cannot assemble without all the components being present due to polar residues in the TM domain. Hence, physiological fluctuation of the CAR occurs through assembly with the CD3 complex.

This solved certain problems noted above but required a bi-cistronic vector to supply VH-Calpha/VL-Cbeta, which is a drawback with this system.

WO 2016/187349 describes an approach which connects a binding domain to CD3 components, typically CD3 epsilon. This can to some extent be expressed by itself so some of the physiological control is lost.

The very first CAR designs proposed by Becker and Gross et al involved incorporation of VH into TCR or VH/VL into the TCR by replacement of alpha or beta variable regions with the VH/VL.

Becker et al. 1989 (Cell, Vol. 58, pages 911 to 921) discloses the fusion of a heavy chain from a digoxin monoclonal antibody to a TCR-Ca molecule. The molecule disclosed in Becker comprises only the heavy chain (V_{H}) fused to the constant region of a TCR-Cα. There is no mention of pre-Tα anywhere in this document.

Gross at al. 1989 (Proc. Natl. Acad. Sci. USA, Vol. 86, pages 10024 to 10028) discloses individual fusions of single V_{H} or V_{L} chains to TCR-Ca or TCR-C polypeptides. In particular, FIG. 1 at page 10025 of Gross et al shows the constructs made. These are discussed in the ‘Results’ section at page 10025 (right-column, last paragraph) where the importance of making single-combination fusions is explained.

A different format of CAR has been described whereby a binding domain is connected directly to a component of CD3 such that the binder-CD3 assembles into the TCR complex. This means that upon recognition of target antigen, the full CD3 complex is engaged in activation. Further, since the CD3/TCR complex only assembles and is expressed if all the elements are expressed, the CAR benefits from physiological fluctuations in TCR expression. WO 2016/187349 (TCR2, Inc.) discloses compositions and methods for TCR reprogramming using fusion proteins. In this approach, an antigen binding domain such as an anti-CD19 scFv is linked to a CD3 polypeptide such as CD3ε, and these are incorporated into a “re-programmed TCR” (see FIG. 1 of WO 2016/187349). This CD3 CAR approach however is limited in that the CAR must compete for CD3 complex.

The present invention seeks to overcome problem(s) associated with the prior art.

### SUMMARY OF THE INVENTION

During T-cell development in the thymus, the TCRβ chain undergoes rearrangement. The rearranged β chain polypeptide then pairs with the pre-Tα polypeptide. This is considered a “holding” pairing during the time whilst the α chain undergoes rearrangement. Once the α chain rearrangement is complete, the rearranged α chain pairs with the rearranged β chain, and the cell may proceed to positive and/or negative selection in the usual manner.

The present inventors have developed a polypeptide which comprises an antigen binding domain with the ectodomain of pre-Tα. When expressed in a cell, the polypeptide pairs with the endogenous TCRβ chain.

The use of pre-Tα polypeptide is advantageous for several reasons. Firstly, the polypeptide of the invention comprising pre-Tα displaces or replaces the endogenous pre-Tα chain in pairing with the endogenous TCRβ chain. This results in a very clean CD3/CDR complex, because there are no complications arising from endogenous TCRα-TCRβ paired complexes. Therefore, any naturally occurring complex which might have been formed by a TCRα/TCRβ pairing is advantageously avoided, and the CD3/TCR complexes formed are comprised of, more suitably consist of, the pre-Tα containing the polypeptide of the invention paired with endogenous TCRβ.

Moreover, pre-Tα has no Z chain (zeta chain) and so alternative or additional signalling elements can be joined to the endoplasmic tail of pre-Tα (for example by protein fusion) with fewer or no complications compared to other molecules.

Thus, in one aspect, the invention relates to a polypeptide comprising

(i) an antigen binding domain

(ii) a pre-T-alpha ectodomain, and

(iii) a transmembrane domain.

Ectodomain has its natural meaning. The person skilled in the art can determine the ectodomain of for example pre-T-alpha as a matter of routine. For example, the TMHMM algorithm can be used to locate position of the transmembrane helices. The TMHMM algorithm is a membrane protein topology prediction method.

In case any guidance is needed, the reference sequence for pre-T-alpha provided in SEQ ID NO: 1 below is 281 amino acids long; the ectodomain of pre-T-alpha is 123 amino acids (SEQ ID NO: 3). The address of the mature ectodomain on reference sequence SEQ ID NO: 1 is aa 24 to 146.

Suitably the pre-T-alpha ectodomain comprises the amino acid sequence of SEQ ID NO: 3, or comprises an amino acid sequence having at least 80% sequence identity to SEQ ID NO: 3. More suitably the pre-T-alpha ectodomain consists of the amino acid sequence of SEQ ID NO: 3, or consists of an amino acid sequence having at least 80% sequence identity to SEQ ID NO: 3.

In one aspect, the invention relates to a polypeptide as described above which further comprises a signal sequence, wherein said signal sequence is covalently attached to the N-terminal end of the antigen binding domain.

Suitably said signal sequence may be any signal sequence (signal peptide) such that when it is expressed in a cell, such as a T-cell, the nascent protein is directed to the endoplasmic reticulum and subsequently to the cell surface, where it is expressed. The signal peptide may contain a stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal sequence may be a kappa-chain signal sequence.

Suitably said signal sequence comprises amino acid sequence selected from the group consisting of: SEQ ID NO: 2 and SEQ ID NO: 7, or comprises an amino acid sequence having at least 80% sequence identity to amino acid sequence selected from the group consisting of: SEQ ID NO: 2 and SEQ ID NO: 7. More suitably said signal sequence consists of amino acid sequence selected from the group consisting of: SEQ ID NO: 2 and SEQ ID NO: 7, or consists of an amino acid sequence having at least 80% sequence identity to amino acid sequence selected from the group consisting of: SEQ ID NO: 2 and SEQ ID NO: 7.

The transmembrane domain is the sequence of a classical CAR that spans the membrane. The transmembrane domain may comprise a hydrophobic alpha helix.

The transmembrane domain may comprise one or more ionisable residues. The one or more ionisable residues may, for example, be selected from the following group: Asparagine, Glutamine, Lysine and Arginine. In the TCR receptor complex, ionisable residues in the transmembrane domain of each subunit for a polar network of interactions which hold the complex together. The or each ionisable residue in the transmembrane domain of the polypeptide of the invention may be involved in forming a complex with TCRbeta.

The transmembrane domains of the TCR alpha chain (FIG. 1a) and pre-T-alpha (FIG. 3) comprise an ionisable arginine residue and an ionisable lysine residue. The transmembrane domain of the polypeptide of the present invention may comprise an ionisable arginine residue and/or an ionisable lysine residue at an equivalent position to TCRalpha chain transmembrane domain or pre-T-alpha transmembrane domain. In one embodiment the polypeptide comprises a TCRalpha chain transmembrane domain.

In another embodiment suitably said transmembrane domain comprises a pre-T-alpha transmembrane domain. In this embodiment more suitably the transmembrane domain consists of the pre-T-alpha transmembrane domain. More suitably said pre-T-alpha transmembrane domain comprises the amino acid sequence of SEQ ID NO: 4, or comprises an amino acid sequence having at least 80% sequence identity to SEQ ID NO: 4. Even more suitably said pre-T-alpha transmembrane domain consists of the amino acid sequence of SEQ ID NO: 4, or consists of an amino acid sequence having at least 80% sequence identity to SEQ ID NO: 4.

In one aspect, the invention relates to a polypeptide as described above further comprising an endodomain covalently linked to the C-terminal end of the transmembrane domain.

Endodomain has its natural meaning. The person skilled in the art can determine the endodomain of for example pre-T-alpha or CD28 as a matter of routine. In case any guidance is needed, the reference sequence for pre-T-alpha provided in SEQ ID NO: 1 below is 281 amino acids long; the endodomain of pre-T-alpha is 114 amino acids (SEQ ID NO: 5). The address of the endodomain on reference sequence SEQ ID NO: 1 is aa 168 to 281. In case any additional guidance is needed, the endodomain of CD28 is provided as SEQ ID NO: 11; the endodomain of 41BB is provided as SEQ ID NO: 16; the endodomain of CD148 is provided as SEQ ID NO: 24.

Suitably said endodomain comprises a costimulation domain. Most suitably the costimulation domain is covalently linked to the transmembrane domain at the C-terminal end of the transmembrane domain.

In one embodiment suitably the endodomain comprises, or further comprises, a pre-T-alpha endodomain. More suitably said polypeptide further comprises a costimulation domain covalently linked to the pre-T-alpha endodomain. In this embodiment suitably the costimulation domain is covalently linked to the pre-T-alpha endodomain at the C-terminal end of the pre-T-alpha endodomain.

Suitably said costimulation domain is an Ig family costimulation domain.

Suitably said costimulation domain is a TNF family costimulation domain.

Suitably said costimulation domain comprises one or more of 41BB, OX40, CD27, or TIGR; or CD28 or ICOS costimulation domains.

Suitably the pre-T-alpha domain comprises a deletion relative to the wild-type pre-T-alpha sequence. Suitably said deletion is a deletion of the endodomain of the pre-T-alpha sequence.

In one embodiment suitably pre-T-alpha endodomain is replaced with a co-stimulation domain. Suitably the pre-T-alpha endodomain is deleted and a co-stimulation domain is covalently attached to the polypeptide at the location of the deleted pre-T-alpha endodomain. Suitably the first amino acid of the co-stimulation domain is located at the position of the first amino acid of the deleted pre-T-alpha endodomain; of course the skilled reader will appreciate that these two domains need not be the same length—this guidance is intended to indicate the point of ‘insertion’ or fusion/attachment of the costimulation domain. Suitably the costimulation domain is either a Ig family (e.g. 41BB, OX40, CD27, or TIGR) costimulation domain or a TNF family (e.g. CD28 or ICOS) costimulation domain.

Suitably said costimulation domain comprises a CD28 or 41BB costimulation domain. Most suitably said costimulation domain comprises a CD28 costimulation domain.

In some embodiments the polypeptide of the invention is inducible (or ‘activatable’). In this context ‘inducible’ refers to the activity of the polypeptide (rather than the expression). Thus the polypeptide will typically be present/expressed and the activity will be induced or suppressed via ligation or dissociation of a separate docking polypeptide via the heterodimerisation domain. Thus the heterodimerisation domain is a domain allowing the activity of the polypeptide to be controlled such as to be induced (activated) or suppressed (inactivated). Suitably when the polypeptide of the invention comprises heterdimerisation domain, the cognate docking polypeptide such as an activator (switch-on) or an inhibitor (switch-off) may also be supplied. Thus in one embodiment a docking polypeptide is provided which comprises a second heterodimerisation domain, the cognate partner of the heterodimerisation domain of the polypeptide of the present invention. The docking polypeptide may be an activator (switch-on) or may be an inhibitor (switch-off). Thus suitably the heterodimerisation domain is a polypeptide domain capable of receiving activation and/or inhibition signal from the docking polypeptide. Exemplary inducible formats may comprise: antigen binding domain-PreTalpha domain-TetR/2A/Tip-CD45 or Tip-CD148 (switch on—assembles in absence of Tet/Mino); and/or antigen binding domain-PreTalpha domain-FKBP12/2A/FRB-CD45 (switch off) and/or antigen binding domain-PreTalpha domain—FKBP12/2A/FRB-dCD148 (switch on—assembles in presence of Rapamycin/Rapalogues). Reference is made to FIGS. 7a and 7b. Thus in one embodiment the invention relates to provision of induction/activation docking polypeptides or constructs in trans. For example when the polypeptide of the invention comprises a heterodimerisationdomain comprising FRB, suitably a docking polypeptide is supplied comprising cognate heterodimerisation domain FKBP12-dCD128 or FKBP12-dCD45, or vice versa (i.e. when the polypeptide of the invention comprises an heterodimerisation domain comprising FKBP12, suitably a second polypeptide is supplied comprising cognate heterodimerisation domain FRB-dCD128 or FRB-dCD45). The same applies for other heterodimerisation domains such as TetR/Tip as noted above, or for any other suitable heterodimerisation domains known in the art.

The heterodimerisation domains may be capable of dimerising only in the presence of an agent i.e. a separate molecule acting as an “inducer” of dimerization.

The macrolides rapamycin and FK506 act by inducing the heterodimerization of cellular proteins. Each drug binds with a high affinity to the FKBP12 protein, creating a drug-protein complex that subsequently binds and inactivates mTOR/FRAP and calcineurin, respectively. The FKBP-rapamycin binding (FRB) domain of mTOR has been defined and applied as an isolated 89 amino acid protein moiety that can be fused to a protein of interest. Rapamycin can then induce the approximation of FRB fusions to FKBP12 or proteins fused with FKBP 12. In the context of the present invention, one of the polypeptide and the docking polypeptide may comprise FRB or a variant thereof and the other may comprise FKBP12 or a variant thereof.

The polypeptide and docking polypeptide may be capable of dimerising only in the absence of an agent i.e. a separate molecule may act as an “inhibitor” of dimerization. In this embodiment, dimerization between the first and second dimerization domains is disrupted by the presence of an agent.

The agent may be a molecule, for example a small molecule, which is capable of specifically binding to the first dimerisation domain or the second dimerisation domain at a higher affinity than the binding between the first dimerisation domain and the second dimerisation domain.

For example, the binding system may be based on a peptide:peptide binding domain system. The first or second binding domain may comprise the peptide binding domain and the other binding domain may comprise a peptide mimic which binds the peptide binding domain with lower affinity than the peptide. The use of peptide as agent disrupts the binding of the peptide mimic to the peptide binding domain through competitive binding. The peptide mimic may have a similar amino acid sequence to the “wild-type” peptide, but with one of more amino acid changes to reduce binding affinity for the peptide binding domain.

In this embodiment, the agent may bind the first binding domain or the second binding domain with at least 10, 20, 50, 100, 1000 or 10000-fold greater affinity than the affinity between the first binding domain and the second binding domain.

Small molecules agents which disrupt protein-protein interactions have long been developed for pharmaceutical purposes (reviewed by Vassilev et al; Small-Molecule Inhibitors of Protein-Protein Interactions ISBN: 978-3-642-17082-9). The proteins or peptides whose interaction is disrupted (or relevant fragments of these proteins) can be used as the activation domain/cognate partner and the small molecule may be used as the agent.

A list of proteins/peptides whose interaction is disruptable using an agent such as a small molecule is given in the Table below:

| Interacting Protein 1 | Interacting Protein 2 | Inhibitor of PPI |
|---|---|---|
| P53 | MDM2 | Nutlin |
| Anti-apoptotic Bcl2 | Apoptotic Bcl2 | GX015 and |
| member | member | ABT-737 |
| Caspase-3, -7 or -9 | X-linked inhibitor of | DIABLO and |
| | apoptosis protein | DIABLO |
| | (XIAP) | mimetics |
| RAS | RAF | Furano-indene |
| | | derivative |
| FR2-7 | PD2 domain of DVL | FJ9 |
| T-cell factor (TCF) | Cyclic AMP response | ICG-001 |
| | element binding | |
| | protein (CBP) | |

Further information on these PPIs is available from White et al 2008 (Expert Rev. Mol. Med. 10:e8).

Other small molecule systems for controlling the co-localization of peptides are known in the art, for example the Tet repressor (TetR), TetR interacting protein (TIP), tetracycline system.

The Tet operon is a well-known biological operon which has been adapted for use in mammalian cells. The TetR binds tetracycline as a homodimer and undergoes a conformational change which then modulates the DNA binding of the TetR molecules.

Klotzsche et al. (as above), described a phage-display derived peptide which activates the TetR. This protein (TetR interacting protein/TIP) has a binding site in TetR which overlaps, but is not identical to, the tetracycline binding site. Thus TIP and tetracycline compete for binding of TetR.

Thus the heterodimerisation domain on the polypeptide of the invention may be TetR or TIP, and the heterodimerisation domain on the docking polypeptide may be the corresponding, complementary binding partner.

Where TetR or TIP are used, the agent may be tetracycline, doxycycline, minocycline or an analogue thereof. An analogue refers to a variant of tetracycline, doxycycline or minocycline which retains the ability to specifically bind to TetR.

In one aspect, the invention relates to a polypeptide as described above wherein the endodomain comprises, or further comprises, a heterodimerisation domain. Suitably said heterodimerisation domain comprises TetR, Tip, FRB or FKBP12. Most suitably said heterodimerisation domain comprises TetR or FRB.

In one aspect, the invention relates to a polypeptide as described above further comprising a linker anchor domain. Suitably said linker anchor domain is between said transmembrane domain and said activation domain.

In one embodiment when said endodomain comprises a costimulation domain and an activation domain, suitably these are in the order N-costimulation domain-activation domain-C. In another embodiment when said endodomain comprises a costimulation domain and an activation domain, suitably these are in the order N-activation domain-costimulation domain-C.

In one aspect, the invention relates to a polypeptide as described above wherein said antigen binding domain comprises an immunoglobulin light chain variable region (V_{L}) or an immunoglobulin heavy chain variable region (V_{H}).

Suitably said antigen binding domain comprises both an immunoglobulin light chain variable region (V_{L}) and an immunoglobulin heavy chain variable region (V_{H}).

Suitably said antigen binding domain comprises a scFv, a dsscFv, a dAb, or a ligand.

Suitably said antigen binding domain comprises a scFv.

Suitably said antigen binding domain is capable of specifically binding CD19 (FMC63).

Suitably antigen binding domain(s) capable of binding other targets of interest such as tumour associated antigens (TAA's) may be used, such as a scFv capable of binding such TAA's or other target(s).

Suitably said antigen binding domain comprises amino acid sequence of SEQ ID NO: 8.

Suitably said antigen binding domain consists of amino acid sequence of SEQ ID NO: 8.

In one aspect, the invention relates to a polypeptide comprising an amino acid sequence selected from the group consisting of SEQ ID NO: 6, SEQ ID NO: 10, SEQ ID NO: 12, SEQ ID NO: 18, or SEQ ID NO: 25.

Suitably said polypeptide consists of an amino acid sequence selected from the group consisting of SEQ ID NO: 6, SEQ ID NO: 10, SEQ ID NO: 12, SEQ ID NO: 18, or SEQ ID NO: 25.

Suitably said antigen binding domain and said pre-T-alpha domain are covalently linked by a peptide bond. In other words suitably said antigen binding domain and said pre-T-alpha domain are comprised by a single fusion protein. Suitably said antigen binding domain and said pre-T-alpha domain are linked to form a single fusion protein. Suitably each of the domains comprised by the polypeptide of the invention are covalently linked by a peptide bond. In other words suitably each of the domains comprised by the polypeptide of the invention are comprised by a single fusion protein.

In one aspect, the invention relates to a polypeptide comprising in order: N-terminus-antigen binding domain—pre-T-alpha domain—C-terminus. In one embodiment suitably said polypeptide comprises in order: N-terminus—antigen binding domain-pre-T-alpha domain—transmembrane domain—C-terminus. In one embodiment suitably said polypeptide comprises in order: N-terminus—signal peptide—antigen binding domain—a pre-T-alpha domain—C-terminus.

Suitably said polypeptide comprises in order: N-terminus—signal peptide—antigen binding domain—a pre-T-alpha domain—transmembrane domain—C-terminus.

In one aspect, the invention relates to a complex comprising a polypeptide as described above, and a TCRβ polypeptide.

Suitably said complex further comprises one or more CD3 signalling dimers.

Suitably said complex comprises at least one CD3δε dimer, at least one CD3γε dimer, and at least one ζζ dimer (CD3ζζ dimer). Most suitably said complex comprises one CD3δε dimer, one CD3γε dimer, and one ζζ dimer.

Suitably said complex further comprises a polypeptide comprising a CD3ε-costimulation domain fusion.

In one embodiment when said polypeptide as described above comprises a first heterodimerisation domain, said complex further comprises a docing polypeptide comprising a second heterodimerisation domain, capable of binding to the first heterodimerisation domain.

Suitably said complex is a TCR complex. Suitably said complex is located at a plasma membrane. Suitably said plasma membrane is a cell membrane, suitably a mammalian cell membrane, most suitably a human cell membrane. Suitably said complex spans said membrane.

In one aspect, the invention relates to a nucleic acid comprising nucleotide sequence encoding a polypeptide as described above.

In one aspect, the invention relates to a kit comprising
(i) a nucleotide sequence encoding a polypeptide as described above comprising a first heterodimerisation domain; and
(ii) a nucleotide sequence encoding a docking component which comprises a second heterodimerisation domain which heterodimerises with the first heterodimerisation domain of the polypeptide of (i).

In one aspect, the invention relates to a kit comprising
(i) a nucleotide sequence as described above, and
(ii) a nucleotide sequence encoding a CD3ε-costimulation domain fusion protein.

In one embodiment suitably said nucleotide sequence of (i) and said nucleotide sequence of (ii) are provided on the same nucleic acid.

In one embodiment suitably said nucleotide sequence of (i) and said nucleotide sequence of (ii) are provided on different nucleic acids.

In one aspect, the invention relates to vector comprising a nucleotide sequence as described above.

In one aspect, the invention relates to a kit of vectors comprising
(i) a first vector comprising a nucleotide sequence as defined as (i) above; and
(ii) a second vector comprising a nucleotide sequence as defined as (ii) above.

In one aspect, the invention relates to a cell comprising a polypeptide as described above, a complex as described above, or a nucleic acid as described above.

In one aspect, the invention relates to a method for making a cell as defined above, which comprises the step of transducing or transfecting a cell with a nucleotide sequence, a kit of nucleotide sequences, a vector or a kit of vectors as defined above.

In one aspect, the invention relates to a method of treating a subject comprising administering to said subject a cell as described above.

In one aspect, the invention relates to a cell as described above for use in medicine.

In one aspect, the invention relates to a cell as described above for use in the treatment of cancer.

In one aspect, the invention relates to a cell as defined above in the manufacture of a medicament for treating cancer.

Optionally the polypeptide of the invention may comprise an inert endodomain anchor. Suitably this is in the C-terminal part of the polypeptide. Suitably this is located C-terminal to the transmembrane domain. Most suitably this is covalently joined to the C-terminal end of the transmembrane domain.

### DETAILED DESCRIFIION

The present inventors have used pre-T-alpha to re-direct the CD3 complex to a target antigen. An antigen recognition domain (antigen binding domain) (e.g. a scFv) is attached to the amino-terminus of the pre-T-alpha chain. Thus in a broad aspect the invention relates to an antigen binding domain attached to Pre-T-alpha.

Suitably the endodomain is truncated after the polar anchor to remove any Golgi retention signals.

This has advantages over the known designs such as of Gross et al (ibid.) since advantageously only a monocistronic transgene is required. In the art, the mature TCR alpha/beta chains need to pair up both the constant and variable regions so replacing TCR alpha also required cognate replacement of TCR beta. By contrast, the invention exploits the inventors' insight that the pre-T-alpha is invariant and can pair up on its own with the TCR beta chain.

The polypeptides of the present invention do not signal in themselves—so even small amount of unexpressed protein do not signal on their own which is an advantage compared to the known WO2016/187349 (TCR2 Inc.) format.

### Further Advantages

Considering Becker et al. 1989 (Cell, Vol. 58, pages 911 to 921), this document discloses the fusion of a heavy chain from a digoxin monoclonal antibody to a TCR-Cα molecule. The molecule disclosed in Becker comprises only the heavy chain (V_{H}) fused to the constant region of a TCR. In contrast, the present invention provides a convenient way of delivering both V_{L} and V_{H} chains in a single polypeptide chain, fused to pre-Tα thereby enabling pairing of the endogenous TCRβ and reconstitution of a productive CD3/TCR complex bearing both the heavy and light chains of an immunoglobulin for recognition.

Considering Gross at al. 1989 (Proc. Natl. Acad. Sci. USA, Vol. 86, pages 10024 to 10028), this document teaches that the two key immunoglobulin chains (i.e. V_{H} and V_{L}) have to be separately encoded. Therefore, following the teachings in this document entails carrying the burden of having to produce a pair of polypeptide chains from a pair of open reading frames. By contrast, the present invention conveniently provides the delivery of both the heavy and light chains as a single polypeptide fused to pre-Tα. This improves the simplicity, and also reduces the labour and cost of the system.

A further benefit of the invention compared to prior approaches such as WO 2016/187349 (TCR2, Inc.) is that the invention enables incorporation of co-stimulatory domains.

The invention advantageously demonstrates the practical application of using pre-Tα in place of a TCRα chain. This results in only the intended binder being present in the CD3/TCR complex, and advantageously avoids any naturally occurring TCR binder (e.g. formed from the endogenous TCRα and TCRβ chains) being present in CD3/TCR complexes at the same time. Thus, it is a further benefit of the invention that this source of possible confounding binding/targeting activity is advantageously avoided.

Since CAR T-cells are autonomous, it can be desirable to be able to remotely control CAR T-cells with systemically admitted small molecules. It can sometimes be useful to have the small molecule either switch the CAR on or off depending on the clinical context. The invention allow CARs to be switched on or off with a small molecule either through assembly of heterdimerization domains or through disruption of protein-protein interactions. This is mediated by the activation domains (and their cognate partners) described herein.

Pre-Tα/Pre-Talpha/pTα

The pre-TCR-alpha (PTCA) is expressed early during T-cell development after TCR Beta gene re-arrangement but before TCR alpha gene re-arrangement. It can pair with the beta chain in the absence of a variable region. It incorporates with the CD3/TCR complex and enhances signalling.

Attachment of a binding domain to the PTCA couples the TCR to the CAR with a monocistronic transgene and is superior to known approaches which seek to achieve coupling via CD3 elements.

The pTalpha gene encodes a transmembrane protein that belongs to the Ig superfamily. Pre-Talpha contains a cytoplasmic tail that has no essential function in signal transduction.

The pTalpha receptor (pre-TCR) minimally consists of the TCR beta chain and the disulfide-linked preTalpha chain in association with signal-transducing CD3 molecules. This rescues from programmed cell death cells with productive TCR beta rearrangements.

The pre-TCR induces expansion and differentiation of these cells such that they become TCR alpha beta bearing CD4+8+ thymocytes, which express only a single TCR beta chain and then either die of neglect or—upon TCR-ligand interaction—undergo either positive or negative selection.

Experiments in pT alpha gene-deficient mice show that the pre-TCR has a crucial role in maturation as well as allelic exclusion of alpha beta T cells but is not required for the development of gamma delta-expressing cells. The function of the pre-TCR cannot be fully assumed by an alpha beta TCR that is expressed abnormally early in T cell development.

### Reference Sequence

Suitably all sequences herein are discussed with reference to human pre-T-alpha, most suitably human wild-type pre-T-alpha.

It may be helpful to refer to the sequence of GenBank accession No. AAF21890.1 (partial sequence).

There are different isoforms of human pre T alpha, for example isoforms 1 to 4; for example isoforms CRA_a, CRA_b and CRA_c. Unless otherwise apparent from the context, alternate isomers find application in the invention. Different isomers, or domains/sequences from different isomers, may be selected by the skilled person according to their needs when working the invention. Composite polypeptides comprising one domain/sequence from a first isomer and a second or further domain/sequence from a second or further isomer may be constructed.

An exemplary sequence is pre T-cell antigen receptor alpha isoform 2 precursor [Homo sapiens] NCBI Reference Sequence: NP_612153.2 (also known as pre T-cell antigen receptor alpha, isoform CRA_a [Homo sapiens] GenBank: EAX04107.1-100% identical 281 aa sequence):

MAGTWLLLLLALGCPALPTGVGGTPFPSLAPPIMLLVDGKQQMVVVCLVL
DVAPPGLDSPIWFSAGNGSALDAFTYGPSPATDGTWTNLAHLSLPSEELA
SWEPLVCHTGPGAEGHSRSTQPMHLSGEASTARTCPQEPLRGTPGGALWL
GVLRLLLFKLLLFDLLLTCSCLCDPAGPLPSPATTTRLRALGSHRLHPAT
ETGGREATSSPRPQPRDRRWGDTPPGRKPGSPVWGEGSYLSSYPTCPAQA
WCSRSALRAPSSSLGAFFAGDLPPPLQAGAA

GenBank is a sequence database as described in Benson, D. et al, Nucleic Acids Res. 45(D1):D37-D42 (2017). In more detail, GenBank is as administered by the National Center for Biotechnology Information, National Library of Medicine, 38A, 8N805, 8600 Rockville Pike, Bethesda, Md. 20894, USA. Suitably the current version of sequence database(s) are relied upon. Alternatively, the release in force at the date of filing is relied upon. For the avoidance of doubt, NCBI-GenBank Release 223.0 (15 Dec. 2017) is relied upon.

For the avoidance of doubt, the most preferred exemplary pre-T-alpha sequence is presented below:

Pre-T-alpha (full amino acid seq)
SEQ ID NO: 1
MAGTWLLLLLALGCPALPTGVGGTPFPSLAPPIMLLVDGKQQMVVVCLVL
DVAPPGLDSPIWFSAGNGSALDAFTYGPSPATDGTWTNLAHLSLPSEELA
SWEPLVCHTGPGAEGHSRSTQPMHLSGEASTARTCPQEPLRGTPGGALWL
GVLRLLLFKLLLFDLLLTCSCLCDPAGPLPSPATTTRLRALGSHRLHPAT
ETGGREATSSPRPQPRDRRWGDTPPGRKPGSPVWGEGSYLSSYPTCPAQA
WCSRSALRAPSSSLGAFFAGDLPPPLQAGAA

In more detail, SEQ ID NO: 1 is a contiguous sequence comprised of several domains which may be independently useful. These domains have exemplary sequences as follows:

Signal sequence:
SEQ ID NO: 2
MAGTWLLLLLALGCPALPTGVGG
Ectodomain:
SEQ ID NO: 3
TPFPSLAPPIMLLVDGKQQMVVVCLVLDVAPPGLDSPIWFSAGNGSALDA
FTYGPSPATDGTWTNLAHLSLPSEELASWEPLVCHTGPGAEGHSRSTQPM
HLSGEASTARTCPQEPLRGTPGG
Transmembrane:
SEQ ID NO: 4
ALWLGVLRLLLFKLLLFDLLL
Endodomain:
SEQ ID NO: 5
TCSCLCDPAGPLPSPATTTRLRALGSHRLHPATETGGREATSSPRPQPRD
RRWGDTPPGRKPGSPVWGEGSYLSSYPTCPAQAWCSRSALRAPSSSLGAF
FAGDLPPPLQAGAA

When particular amino acid residues are referred to herein using numeric addresses, the numbering is taken with reference to the wild type pre-T alpha amino acid sequence (or to the polynucleotide sequence encoding same if referring to nucleic acid) as shown above (e.g. SEQ ID NO: 1). This sequence is to be used as is well understood in the art to locate the feature/residue of interest. This is not always a strict counting exercise-attention must be paid to the context. For example, if the protein of interest is of a slightly different length, then location of the correct residue in that sequence may require the sequences to be aligned and the equivalent or corresponding residue picked. This is well within the ambit of the skilled reader.

Mutating has it normal meaning in the art and may refer to the substitution or truncation or deletion or addition of one or more residues, motifs or domains. Mutation may be effected at the polypeptide level, for example, by synthesis of a polypeptide having the mutated sequence, or may be effected at the nucleotide level, for example, by making a polynucleotide encoding the mutated sequence, which polynucleotide may be subsequently translated to produce the mutated polypeptide.

Sequence Variation

The polypeptides described herein may comprise sequence changes relative to the wild type sequence. Specifically the polypeptides described herein may comprise sequence changes at sites which do not significantly compromise the function or operation of the polypeptides described herein. The sequence changes may be at the polypeptide or the nucleotide level.

Polypeptides include variants produced by introducing any type of additional alterations (for example, insertions, deletions, or substitutions of amino acids; changes in glycosylation states; changes that affect refolding or isomerizations, three-dimensional structures, or self-association states), which can be deliberately engineered. The variant may have alterations which produce a silent change and result in a functionally equivalent polypeptide. Deliberate amino acid substitutions may be made on the basis of similarity in polarity, charge, solubility, hydrophobicity, hydrophilicity and the amphipathic nature of the residues as long as the structure or conformation of the polypeptide is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the table below. Amino acids in the same block in the second column and suitably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

In considering what mutations, substitutions or other such changes might be made relative to the wild type sequence, retention of the structure, conformation and/or function of the polypeptide is important, most importantly the function. Typically conservative amino acid substitutions would be less likely to adversely affect the function.

Suitably some residues are not mutated for example some residues within the transmembrane domain are well known to be important for functional pairing of signalling dimers in the TCR complex by salt-bridge-type amino acid associations as explained above with reference to FIGS. 1 to 3. Thus suitably basic and acidic residues in the trans-membrane (TM) domains of the TCR and CD3 subunits described herein are not mutated. Similar considerations may apply to other residues in the polypeptides described herein as will be apparent to the skilled worker.

Sequence Identity

For precision, sequence relationships have been discussed relative to a reference sequence, such as SEQ ID NO: 1 (wild type human pre-T-alpha). Clearly the reference sequence will be different when other proteins are discussed e.g. CD19, CD148, FRB etc. Suitably the reference sequence for any such proteins is the wild type sequence, most suitably the wild type human sequence, unless other sequence is noted or provided herein. If multiple wild type human sequences are known, the reference sequence is suitably the most common version available.

It may be desired to consider sequence relationships in terms of sequence identity. Sequence comparisons can be conducted by eye or, more usually, with the aid of readily available sequence comparison programs. These publicly and commercially available computer programs can calculate percent homology (such as percent identity) between two or more sequences.

Percent identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example in an otherwise identical pair of sequences, one insertion or deletion will cause the following amino acid residues to be put out of alignment, thus potentially resulting in a large reduction in percent homology (percent identity) when a global alignment (an alignment across the whole sequence) is performed. Consequently, most sequence comparison methods are designed to produce optimal alignments that take into consideration possible insertions and deletions without penalising unduly the overall homology (identity) score. This is achieved by inserting “gaps” in the sequence alignment to try to maximise local homology/identity.

These more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package, FASTA (Altschul et al., 1990, J. Mol. Biol. 215:403-410) and the GENEWORKS suite of comparison tools.

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62. Once the software has produced an optimal alignment, it is possible to calculate percent homology, preferably percent sequence identity. The software typically does this as part of the sequence comparison and generates a numerical result.

Suitably the polypeptide(s) or polypeptide domain(s) discussed herein has at least 80% sequence identity to the reference sequence (e.g. for Pre-T-alpha the reference sequence is SEQ ID NO: 1), more suitably 85%, more suitably 88%, more suitably 90%, more suitably 92%, more suitably 94%, more suitably 95%, more suitably 96%, more suitably 97%, more suitably 98%, more suitably 99% identity to the reference sequence (for pre-T-alpha the reference sequence is SEQ ID NO: 1).

When only a domain is taken, the domain sequence may be shorter than the reference sequence. For example when the ectodomain of pre-T-alpha is considered, this is 123 amino acids (SEQ ID NO: 3). The importance is to consider the sequence identity against the corresponding part or section of the reference sequence, rather than decreasing the sequence identity score for ‘missing’ amino acids. Therefore suitably sequence identity is suitably considered across the whole length of the query sequence against the corresponding section of the reference sequence. Therefore the sequence identity of SEQ ID NO: 3 compared to the corresponding section of the reference sequence (SEQ ID NO: 1) is 100%. Clearly any mutations (e.g. substitutions, deletions, additions etc) within the domain or section being considered will lead to a correspondingly lower sequence identity score as is conventional in the field.

In all discussions of sequence identity, it will be noted that SEQ ID NO: 1 is 281 amino acids in length. Therefore each single substitution is equivalent to 0.35587% change in identity if all 281 amino acids are considered. The above values are given to nearest whole percentage point and should be understood accordingly given that it is not possible to substitute partial amino acids within a polypeptide sequence. Clearly when fewer than 281 amino acids are considered (for example when only a domain of a polypeptide is taken and the reference sequence is longer than that domain, or when a reference sequence for protein other than pre-T-alpha/SEQ ID NO:1 is used which may be shorter or longer than 281 amino acids) then each single amino acid substitution may correspond to a greater or lesser % change in identity; the skilled reader can interpret the values accordingly given that it is not possible to substitute partial amino acids within a polypeptide sequence.

Sequence variants having particular sequence identity levels are used in the invention always provided that the polypeptide retains the function by reference to the reference sequence such as wild type human pre-T-alpha.

Polypeptide function may be easily tested using the methods as set out herein, such as in the examples section, for example in order to verify that the peptides assemble correctly and/or transmit signal when appropriately stimulated. Suitably the polypeptides should retain the function of supporting T cell survival when appropriately stimulated. Suitably the polypeptides should retain the function of supporting T cell proliferation when appropriately stimulated. Thus, provided that the polypeptide retains its function, which can be easily tested as set out herein (see examples section for further guidance), sequence variations may be made in the polypeptide relative to the wild type reference sequence.

For example, a mutated polypeptide may be tested in in vitro assays with SupT1-GFP cells expressing CD19 (low/high) to check function in supporting cytolysis of target cells. IFN-gamma and/or IL2 release may also be assayed.

Polypeptides and Domains

An exemplary polypeptide according to the present invention is fmc63-dPreTalpha (FIG. 5b). This polypeptide is shown as SEQ ID NO: 6:

METDTLLLWVLLLWVPGSTGDIQMTQTTSSLSASLGDRVTISCRASQDIS
KYLNWYQQKPDGTVKLLIYHTSRLHSGVPSRFSGSGSGTDYSLTISNLEQ
EDIATYFCQQGNTLPYTFGGGTKLEITKAGGGGSGGGGSGGGGSGGGGSE
VKLQESGPGLVAPSQSLSVTCTVSGVSLPDYGVSWIRQPPRKGLEWLGVI
WGSETTYYNSALKSRLTIIKDNSKSQVFLKMNSLQTDDTAIYYCAKHYYY
GGSYAMDYWGQGTSVTVSSDPTPFPSLAPPIMLLVDGKQQMVVVCLVLDV
APPGLDSPIWFSAGNGSALDAFTYGPSPATDGTWTNLAHLSLPSEELASW
EPLVCHTGPGAEGHSRSTQPMHLSGEASTARTCPQEPLRGTPGGALWLGV
LRLLLFKLLLFDLLLQRALVLRRKRKRMTDPTRR

In more detail, SEQ ID NO: 6 (fmc63-dPreTalpha (FIG. 5b)) is a contiguous sequence comprised of several domains which may be independently useful in different combination(s) in other polypeptides according to the present invention and/or may be individually exchanged for variant(s) as described herein. These domains have exemplary sequences as follows:

Signal sequence
SEQ ID NO: 7
METDTLLLWVLLLWVPGSTG
scFv_Fmc63
SEQ ID NO: 8
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITKAGGGGSGGGGSGGGGSGGGGSEVKLQESGPGLVAPSQSLSVT
CTVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVSSD
P
Ectodomain derived from pre-T-alpha
SEQ ID NO: 3
TPFPSLAPPIMLLVDGKQQMVVVCLVLDVAPPGLDSPIWFSAGNGSALDA
FTYGPSPATDGTWTNLAHLSLPSEELASWEPLVCHTGPGAEGHSRSTQPM
HLSGEASTARTCPQEPLRGTPGG
Transmembrane derived from pre-T-alpha
SEQ ID NO: 4
ALWLGVLRLLLFKLLLFDLLL
Truncated inert endodomain anchor derived from
CD19
SEQ ID NO: 9
QRALVLRRKRKRMTDPTRR

An exemplary polypeptide according to the present invention is fmc63-PreTalpha-CD28 (FIG. 6a). This has the endodomain of pTalpha replaced with a CD28 costimulation domain. This polypeptide is shown as SEQ ID NO:10:

METDTLLLWVLLLWVPGSTG
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITKAGGGGSGGGGSGGGGSGGGGSEVKLQESGPGLVAPSQSLSVT
CTVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVSSD
PTPFPSLAPPIMLLVDGKQQMVVVCLVLDVAPPGLDSPIWFSAGNGSALD
AFTYGPSPATDGTWTNLAHLSLPSEELASWEPLVCHTGPGAEGHSRSTQP
MHLSGEASTARTCPQEPLRGTPGGALWLGVLRLLLFKLLLFDLLLRSKRS
RLLHSDYMNMTPRRPGPTRKHYQPYAPPRDFAAYRS

In more detail, SEQ ID NO: 10 (fmc63-PreTalpha-CD28 (FIG. 6a)) is a contiguous sequence comprised of several domains which may be independently useful in different combination(s) in other polypeptides according to the present invention and/or may be individually exchanged for variant(s) as described herein. These domains have exemplary sequences as follows:

Signal sequence:
SEQ ID NO: 7
METDTLLLWVLLLWVPGSTG
scFv_Fmc63:
SEQ ID NO: 8
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITKAGGGGSGGGGSGGGGSGGGGSEVKLQESGPGLVAPSQSLSVT
CTVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVSSD
P
Ectodomain derived from pre-T-alpha:
SEQ ID NO: 3
TPFPSLAPPIMLLVDGKQQMVVVCLVLDVAPPGLDSPIWFSAGNGSALDA
FTYGPSPATDGTWTNLAHLSLPSEELASWEPLVCHTGPGAEGHSRSTQPM
HLSGEASTARTCPQEPLRGTPGG
Transmembrane derived from pre-T-alpha:
SEQ ID NO: 4
ALWLGVLRLLLFKLLLFDLLL
Endodomain CD28:
SEQ ID NO: 11
RSKRSRLLHSDYMNMTPRRPGPTRKHYQPYAPPRDFAAYRS

An exemplary polypeptide according to the present invention is fmc6A-PreTalpha-CD28 (FIG. 6b). This has the endodomain of pTalpha replaced with a CD28 costimulation domain. This polypeptide is shown as SEQ ID NO:12:

METDTLLLWVLLLWVPGSTGDIQMTQTTSSLSASLGDRVTISCRASQDIS
KYLNWYQQKPDGTVKLLIYHTSRLHSGVPSRFSGSGSGTDYSLTISNLEQ
EDIATYFCQQGNTLPYTFGGGTKLEITKAGGGGSGGGGSGGGGSGGGGSE
VKLQESGPGLVAPSQSLSVTCTVSGVSLPDYGVSWIRQPPRKGLEWLGVI
WGSETTYYNSALKSRLTIIKDNSKSQVFLKMNSLQTDDTAIYYCAKHYYY
GGSYAMDYWGQGTSVTVSSDPTPFPSLAPPIMLLVDGKQQMVVVCLVLDV
APPGLDSPIWFSAGNGSALDAFTYGPSPATDGTWTNLAHLSLPSEELASW
EPLVCHTGPGAEGHSRSTQPMHLSGEASTARTCPQEPLRGTPGGALWLGV
LRLLLFKLLLFDLLLRSKRSRLLHSDYMNMTPRRPGPTRKHYQPYAPPRD
FAAYRS

In more detail, SEQ ID NO: 12 (fmc63-PreTalpha-CD28 (FIG. 6b)) is a contiguous sequence comprised of several domains which may be independently useful in different combination(s) in other polypeptides according to the present invention and/or may be individually exchanged for variant(s) as described herein. These domains have exemplary sequences as follows:

Signal sequence:
SEQ ID NO: 7
METDTLLLWVLLLWVPGSTG
scFv_Fmc63:
SEQ ID NO: 8
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITKAGGGGSGGGGSGGGGSGGGGSEVKLQESGPGLVAPSQSLSVT
CTVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVSSD
P
Ectodomain derived from pre-T-alpha:
SEQ ID NO: 3
TPFPSLAPPIMLLVDGKQQMVVVCLVLDVAPPGLDSPIWFSAGNGSALDA
FTYGPSPATDGTWTNLAHLSLPSEELASWEPLVCHTGPGAEGHSRSTQPM
HLSGEASTARTCPQEPLRGTPGG
Transmembrane derived from pre-T-alpha:
SEQ ID NO: 4
ALWLGVLRLLLFKLLLFDLLL
Endodomain CD28:
SEQ ID NO: 11
RSKRSRLLHSDYMNMTPRRPGPTRKHYQPYAPPRDFAAYRS

Co-Stimulatory Constructs In Trans

In one embodiment the invention relates to provision of co-stimulatory constructs in trans. In this embodiment other component(s) of the TCR complex may be modified for example by addition of costimulatory domain(s) to the endodomain(s) of said other components. For example in one embodiment a CD3E fusion is made which suitably has a co-stimulatory molecule different from that included in the polypeptide of the invention. Suitably this fusion has a different type of costimulatory domain (e.g. different family of costimulatory domain) from the type on the polypeptide of the invention—this has the advantage that two types (two families) of costimulatory domain are supplied in trans (i.e. one on the polypeptide of the invention and one on the CD3E polypeptide of this embodiment.) In a preferred embodiment the polypeptide of the invention comprises a costimulation domain comprising 41BB/2A; a further costimulatory construct (e.g. polypeptide) is provided comprising CD3E-CD28. Thus in one embodiment the invention relates to a polypeptide comprising a CD3E domain fused to a costimulation domain.

Suitably said costimulation domain comprises one or more of 41BB, OX40, CD27, or TIGR; or CD28 or ICOS.

Most suitably said costimulation domain comprises CD28.

Suitably said polypeptide as described above comprises the amino acid sequence of SEQ ID NO: 13. More suitably said polypeptide as described above consists of the amino acid sequence of SEQ ID NO: 13.

Also provided is a complex comprising a polypeptide of the invention (as described above comprising an antigen binding domain and a pre-T-alpha domain), and a polypeptide according to the current embodiment (comprising a CD3E domain fused to a costimulation domain).

Also provided is a kit comprising a nucleotide sequence encoding a polypeptide of the invention (as described above comprising an antigen binding domain and a pre-T-alpha domain) and a nucleotide sequence encoding a polypeptide according to the current embodiment (comprising a CD3E domain fused to a costimulation domain).

An exemplary CD3E polypeptide according to the present invention is CD3e-41BB-2A (FIG. 6b). This polypeptide is shown as SEQ ID NO:13:

METDTLLLWVLLLWVPGSTGDGNEEMGGITQTPYKVSISGTTVILTCPQY
PGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWS
KRGRKKLLYIFKQPFMRPVQTTQEEDGCSCRFPEEEEGGCELEGRGSLLT
CGDVEENPGP

In more detail, SEQ ID NO: 13 (CD3e-41BB-2A (FIG. 6b)) is a contiguous sequence comprised of several domains which may be independently useful in different combination(s) in other polypeptides according to the present invention and/or may be individually exchanged for variant(s) as described herein. These domains have exemplary sequences as follows:

Signal sequence:
SEQ ID NO: 7
METDTLLLWVLLLWVPGSTG
Ectodomain CD3e:
SEQ ID NO: 14
DGNEEMGGITQTPYKVSISGTTVILTCPQYPGSEILWQHNDKNIGGDEDD
KNIGSDEDHLSLKEFSELEQSGYYVCYPRGSKPEDANFYLYLRARVCENC
MEMD
Transmembrane CD3e:
SEQ ID NO: 15
VMSVATIVIVDICITGGLLLLVYYWS
Endodomain 41BB:
SEQ ID NO: 16
KRGRKKLLYIFKQPFMRPVQTTQEEDGCSCRFPEEEEGGCEL
2A:
SEQ ID NO: 17
EGRGSLLTCGDVEENPGP

Heterodimerisation Domains in Trans

An exemplary polypeptide according to the present invention comprising heterodimerisation domain (such as TetRB) is fmc63-PreTalpha-TetRB (FIG. 7a). This polypeptide is shown as SEQ ID NO:18:

METDTLLLWVLLLWVPGSTGDIQMTQTTSSLSASLGDRVTISCRASQDIS
KYLNWYQQKPDGTVKLLIYHTSRLHSGVPSRFSGSGSGTDYSLTISNLEQ
EDIATYFCQQGNTLPYTFGGGTKLEITKAGGGGSGGGGSGGGGSGGGGSE
VKLQESGPGLVAPSQSLSVTCTVSGVSLPDYGVSWIRQPPRKGLEWLGVI
WGSETTYYNSALKSRLTIIKDNSKSQVFLKMNSLQTDDTAIYYCAKHYYY
GGSYAMDYWGQGTSVTVSSDPTPFPSLAPPIMLLVDGKQQMVVVCLVLDV
APPGLDSPIWFSAGNGSALDAFTYGPSPATDGTWTNLAHLSLPSEELASW
EPLVCHTGPGAEGHSRSTQPMHLSGEASTARTCPQEPLRGTPGGALWLGV
LRLLLFKLLLFDLLLRSKRSRMSRLDKSKVINSALELLNEVGIEGLTTRK
LAQKLGVEQPTLYWHVKNKRALLDALAIEMLDRHHTHFCPLEGESWQDFL
RNNAKSFRCALLSHRDGAKVHLGTRPTEKQYETLENQLAFLCQQGFSLEN
ALYALSAVGHFTLGCVLEDQEHQVAKEERETPTTDSMPPLLRQAIELFDH
QGAEPAFLFGLELIICGLEKQLKCESGS

In more detail, SEQ ID NO:18 (fmc63-PreTalpha-TetRB (FIG. 7a)) is a contiguous sequence comprised of several domains which may be independently useful in different combination(s) in other polypeptides according to the present invention and/or may be individually exchanged for variant(s) as described herein. These domains have exemplary sequences as follows:

Signal sequence:
SEQ ID NO: 7
METDTLLLWVLLLWVPGSTG
scFv_Fmc63:
SEQ ID NO: 8
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITKAGGGGSGGGGSGGGGSGGGGSEVKLQESGPGLVAPSQSLSVT
CTVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVSSD
P
Ectodomain derived from pre-T-alpha:
SEQ ID NO: 3
TPFPSLAPPIMLLVDGKQQMVVVCLVLDVAPPGLDSPIWFSAGNGSALDA
FTYGPSPATDGTWTNLAHLSLPSEELASWEPLVCHTGPGAEGHSRSTQPM
HLSGEASTARTCPQEPLRGTPGG
Transmembrane derived from pre-T-alpha:
SEQ ID NO: 4
ALWLGVLRLLLFKLLLFDLLL
Linker anchor:
SEQ ID NO: 19
RSKRSR
TetRB:
SEQ ID NO: 20
MSRLDKSKVINSALELLNEVGIEGLTTRKLAQKLGVEQPTLYWHVKNKRA
LLDALAIEMLDRHHTHFCPLEGESWQDFLRNNAKSFRCALLSHRDGAKVH
LGTRPTEKQYETLENQLAFLCQQGFSLENALYALSAVGHFTLGCVLEDQE
HQVAKEERETPTTDSMPPLLRQAIELFDHQGAEPAFLFGLELIICGLEKQ
LKCESGS

In this embodiment suitably a second docking polypeptide is supplied comprising an induction/activation domain in trans, for example when the polypeptide of the invention comprises SEQ ID NO: 18, suitably said docking polypeptide comprises TIP-CD148-2A (FIG. 7a)—SEQ ID NO: 21

MWTWNAYAFAAPSGGGSRKKRKDAKNNEVSFSQIKPKKSKLIRVENFEAY
FKKQQADSNCGFAEEYEDLKLVGISQPKYAAELAENRGKNRYNNVLPYDI
SRVKLSVQTHSTDDYINANYMPGYHSKKDFIATQGPLPNTLKDFWRMVWE
KNVYAIIMLTKCVEQGRTKCEEYWPSKQAQDYGDITVAMTSEIVLPEWTI
RDFTVKNIQTSESHPLRQFHFTSWPDHGVPDTTDLLINFRYLVRDYMKQS
PPESPILVHCSAGVGRTGTFIAIDRLIYQIENENTVDVYGIVYDLRMHRP
LMVQTEDQYVFLNQCVLDIVRSQKDSKVDLIYQNTTAMTIYENLAPVTTF
GKTNGYIAEGRGSLLTCGDVEENPGP

In more detail, SEQ ID NO: 21(TIP-CD148-2A (FIG. 7a)) is a contiguous sequence comprised of several domains which may be independently useful in different combination(s) in other polypeptides according to the present invention and/or may be individually exchanged for variant(s) as described herein. These domains have exemplary sequences as follows:

TIP:
SEQ ID NO: 22
MWTWNAYAFAAP
Linker:
SEQ ID NO: 23
SGGGS
Endodomain CD148:
SEQ ID NO: 24
RKKRKDAKNNEVSFSQIKPKKSKLIRVENFEAYFKKQQADSNCGFAEEYE
DLKLVGISQPKYAAELAENRGKNRYNNVLPYDISRVKLSVQTHSTDDYIN
ANYMPGYHSKKDFIATQGPLPNTLKDFWRMVWEKNVYAIIMLTKCVEQGR
TKCEEYWPSKQAQDYGDITVAMTSEIVLPEWTIRDFTVKNIQTSESHPLR
QFHFTSWPDHGVPDTTDLLINFRYLVRDYMKQSPPESPILVHCSAGVGRT
GTFIAIDRLIYQIENENTVDVYGIVYDLRMHRPLMVQTEDQYVFLNQCVL
DIVRSQKDSKVDLIYQNTTAMTIYENLAPVTTFGKTNGYIA
2A:
SEQ ID NO: 17
EGRGSLLTCGDVEENPGP

An exemplary polypeptide according to the present invention comprising heterodimerisation domain (such as FRB) is fmc63-PreTalpha-FRB (FIG. 7b). This polypeptide is shown as SEQ ID NO: 25:

METDTLLLWVLLLWVPGSTGDIQMTQTTSSLSASLGDRVTISCRASQDIS
KYLNWYQQKPDGTVKLLIYHTSRLHSGVPSRFSGSGSGTDYSLTISNLEQ
EDIATYFCQQGNTLPYTFGGGTKLEITKAGGGGSGGGGSGGGGSGGGGSE
VKLQESGPGLVAPSQSLSVTCTVSGVSLPDYGVSWIRQPPRKGLEWLGVI
WGSETTYYNSALKSRLTIIKDNSKSQVFLKMNSLQTDDTAIYYCAKHYYY
GGSYAMDYWGQGTSVTVSSDPTPFPSLAPPIMLLVDGKQQMVVVCLVLDV
APPGLDSPIWFSAGNGSALDAFTYGPSPATDGTWTNLAHLSLPSEELASW
EPLVCHTGPGAEGHSRSTQPMHLSGEASTARTCPQEPLRGTPGGALWLGV
LRLLLFKLLLFDLLLRSKRSRILWHEMWHEGLEEASRLYFGERNVKGMFE
VLEPLHAMMERGPQTLKETSFNQAYGRDLMEAQEWCRKYMKSGNVKDLLQ
AWDLYYHVFRRISK

In more detail, SEQ ID NO: 25 (fmc63-PreTalpha-FRB (FIG. 7b)) is a contiguous sequence comprised of several domains which may be independently useful in different combination(s) in other polypeptides according to the present invention and/or may be individually exchanged for variant(s) as described herein. These domains have exemplary sequences as follows:

Signal sequence:
SEQ ID NO: 7
METDTLLLWVLLLWVPGSTG
scFv_Fmc63:
SEQ ID NO: 8
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITKAGGGGSGGGGSGGGGSGGGGSEVKLQESGPGLVAPSQSLSVT
CTVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVSSD
P
Ectodomain derived from pre-T-alpha:
SEQ ID NO: 3
TPFPSLAPPIMLLVDGKQQMVVVCLVLDVAPPGLDSPIWFSAGNGSALDA
FTYGPSPATDGTWTNLAHLSLPSEELASWEPLVCHTGPGAEGHSRSTQPM
HLSGEASTARTCPQEPLRGTPGG
Transmembrane derived from pre-T-alpha:
SEQ ID NO: 4
ALWLGVLRLLLFKLLLFDLLL
Linker anchor:
SEQ ID NO: 19
RSKRSR
FRB:
SEQ ID NO: 26
ILWHEMWHEGLEEASRLYFGERNVKGMFEVLEPLHAMMERGPQTLKETSF
NQAYGRDLMEAQEWCRKYMKSGNVKDLLQAWDLYYHVFRRISK

In this embodiment suitably a second docking polypeptide is supplied comprising an induction/activation domain in trans, for example when the polypeptide of the invention comprises SEQ ID NO: 25, suitably said second polypeptide comprises FKBP12-CD148-2A (FIG. 7b)—SEQ ID NO: 27

MLEGVQVETISPGDGRTFPKRGQTCVVHYTGMLEDGKKFDSSRDRNKPFK
FMLGKQEVIRGWEEGVAQMSVGQRAKLTISPDYAYGATGHPGIIPPHATL
VFDVELLKLESGGGSRKKRKDAKNNEVSFSQIKPKKSKLIRVENFEAYFK
KQQADSNCGFAEEYEDLKLVGISQPKYAAELAENRGKNRYNNVLPYDISR
VKLSVQTHSTDDYINANYMPGYHSKKDFIATQGPLPNTLKDFWRMVWEKN
VYAIIMLTKCVEQGRTKCEEYWPSKQAQDYGDITVAMTSEIVLPEWTIRD
FTVKNIQTSESHPLRQFHFTSWPDHGVPDTTDLLINFRYLVRDYMKQSPP
ESPILVHCSAGVGRTGTFIAIDRLIYQIENENTVDVYGIVYDLRMHRPLM
VQTEDQYVFLNQCVLDIVRSQKDSKVDLIYQNTTAMTIYENLAPVTTFGK
TNGYIAEGRGSLLTCGDVEENPGP

In more detail, SEQ ID NO: 27 (FKBP12-CD148-2A (FIG. 7b)) is a contiguous sequence comprised of several domains which may be independently useful in different combination(s) in other polypeptides according to the present invention and/or may be individually exchanged for variant(s) as described herein. These domains have exemplary sequences as follows:

FKBP12:
SEQ ID NO: 28
MLEGVQVETISPGDGRTFPKRGQTCVVHYTGMLEDGKKFDSSRDRNKPFK
FMLGKQEVIRGWEEGVAQMSVGQRAKLTISPDYAYGATGHPGIIPPHATL
VFDVELLKLE
Linker:
SEQ ID NO: 23
SGGGS
Endodomain CD148:
SEQ ID NO: 24
RKKRKDAKNNEVSFSQIKPKKSKLIRVENFEAYFKKQQADSNCGFAEEYE
DLKLVGISQPKYAAELAENRGKNRYNNVLPYDISRVKLSVQTHSTDDYIN
ANYMPGYHSKKDFIATQGPLPNTLKDFWRMVWEKNVYAIIMLTKCVEQGR
TKCEEYWPSKQAQDYGDITVAMTSEIVLPEWTIRDFTVKNIQTSESHPLR
QFHFTSWPDHGVPDTTDLLINFRYLVRDYMKQSPPESPILVHCSAGVGRT
GTFIAIDRLIYQIENENTVDVYGIVYDLRMHRPLMVQTEDQYVFLNQCVL
DIVRSQKDSKVDLIYQNTTAMTIYENLAPVTTFGKTNGYIA
2A;
SEQ ID NO: 17
EGRGSLLTCGDVEENPGP

The T Cell Receptor (TCR) and CD3/T-Cell Receptor Complex

The naturally occurring T cell receptor TCR) recognises peptide fragments presented by MHC molecules and delivers signals which control T cell development and function. The T-cell receptor TCR) complex is composed of several membrane proteins: the TCR alpha/beta chains and the CD3 complex (gamma, delta, epsilon and zeta).

The TCRαβ heterodimer signals ligand binding events to the interior of the cell via the non-covalently associated CD3γε, CD3δϵ, and ζζ dimers which contain tyrosine phosphorylation motifs. Thus, the CD3 elements are incorporated into the CD3 complex as dimers and each contains an endodomain with several immunoreceptor tyrosine based activation motifs (ITAMs).

Assembly is facilitated by basic and acidic residues in the trans-membrane (TM) domains of the TCR and CD3 subunits. These residues form pairwise interactions similar to salt bridges. Thus, the CD3 complex has an elaborate assembly mechanism guided by interacting polar residues in the TM domains which must be shielded from the transmembrane space by correct assembly. Formation of the eight-chain TCRαβ-CD3δε-CD3γε-ζζ complex is therefore dependent on proper placement of three groups of three ionisable TM residues. This is illustrated in FIGS. 1 to 3.

Furthermore, the CD3/TCR complex is under a carefully controlled transcriptional programme such that the TCR is transiently suppressed immediately after T-cell activation. This allows the T-cell to rest before activating again.

Antigen Binding Domain

The antigen-binding domain is the portion of a classical CAR which recognises antigen.

The antigen-binding domain is the part of the polypeptide of the invention which actually binds the target molecule or structure of interest. Thus, the antigen-binding domain can be regarded as the “targeting” part of the polypeptide which actually binds to or associates with the target antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor.

Suitably the antigen is a tumour associated antigen (TAA). The antigen-binding domain used in the present invention may be a domain which is capable of binding a TAA. Exemplary TAA's are indicated in the following table:

| Cancer type | TAA |
|---|---|
| Diffuse Large B-cell | CD19, CD20 |
| Lymphoma | |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic | CD5, CD19 |
| Leukaemia | |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

The antigen-binding domain may comprise a proliferation-inducing ligand (APRIL) which binds to B-cell membrane antigen (BCMA) and transmembrane activator and calcium modulator and cyclophilin ligand interactor (TACI). A CAR comprising an APRIL-based antigen-binding domain is described in WO2015/052538.

Suitably the antigen binding domain specifically binds its target antigen.

Suitably the antigen binding domain is based on or derived from immunorecognition molecule(s) such as the antigen binding portion of an immunoglobulin or antibody. Suitably said antigen binding domain comprises an immunoglobulin or antibody domain. Suitably said antigen binding domain comprises an immunoglobulin light chain variable region (V_{L}) and/or an immunoglobulin heavy chain variable region (V_{H}). Suitably the antigen binding domain may be for example a single-chain variable fragment (scFv).

Thus in one embodiment suitably the antigen binding domain comprises, or consists of, a scFv. In other embodiments, the antigen binding domain comprises a light chain and a heavy chain of an amino acid sequence provided herein, or a functional fragment thereof, or an amino acid sequence having at least 1, 2 or 3 modifications but not more than 30, 20 or 10 modifications of an amino acid sequence of a light chain or heavy chain variable region provided herein, or a sequence with 80-99% identity, more suitably 95-99% identity, with an amino acid sequence provided herein.

Construction of scFv's is well known. Briefly, a scFv is a fusion protein of the variable regions of the heavy (V_{H}) and light chains (V_{L}) of an immunoglobulin. These V_{H} and V_{L} chains are typically connected via a linker peptide of about 10 to about 25 amino acids. The linker is usually rich in glycine for flexibility, and/or rich in serine or threonine for solubility. The linker may connect the N-terminus of the V_{H} to the C-terminus of the V_{L}, or vice versa. scFv's retain the specificity of the original immunoglobulin, despite removal of the constant regions and the introduction of the linker. Any adjustments to the arrangement and/or lengths of the amino acid segments incorporated into the scFv and/or the linker design to retain the specificity of the original immunoglobulin are routine and well within the ambit of the skilled worker.

Co-Stimulation

Full T-cell activation requires more than just TCR/CD3 activation. Co-stimulatory signals promote T-cell proliferation and survival. Thus a costimulation domain means a polypeptide domain capable of promoting T-cell proliferation and/or survival. There are two main types of co-stimulatory signals (costimulation domains): those that belong the Ig family (CD28, ICOS) and the TNF family (OX40,41BB, CD27, GITR etc). Normal T-cells receive co-stimulatory signals from accessory immune cells like dendritic cells. CAR T-cells do not participate in a physiological response so do not typically receive these signals. Incorporation of co-stimulatory signals into CARs has the advantage of overcoming this deficit.

Most suitably a mixture of both types of co-stimulatory signal is needed—so called 3^{rd} generation receptors. Current 3^{rd} generation receptors supply co-stimulatory modules in cis which risks crowding 2^{nd} messenger interactions. Some embodiments of the invention advantageously provide costimulatory domains in trans, which overcomes this issue.

Furthermore, since the pre-T-alpha endodomain does not bear ITAMs (like CD3 epsilon), the inventors had the idea to incorporate co-stimulatory signals into the endodomain of pre-T-alpha, and/or replace the endodomain of pre-T-alpha with other domains such as co-stimulation domain(s).

Elements of control can be brought into this structure such as phosphatases.

CDRs

Suitably the antigen binding domain comprises amino acid sequence of the complementarity determining regions (CDRs) of an immunoglobulin or antibody. Suitably the antigen binding domain comprises all six CDRs of an immunoglobulin or antibody.

Identification of the variable regions/CDRs within an immunoglobulin sequence is a matter of routine for the skilled worker (see for example Kabat et al., Sequences of Proteins of Immunological Interest, 5th Ed. Public Health Service, National Institutes of Health, Bethesda, Md. (1991); (Chothia and Lesk J. Mol. Biol. 196:901-917 (1987)). In more detail, each V region typically comprises three complementarity determining regions (“CDRs”, each of which contains a “hypervariable loop”), and four framework regions. An antibody binding site, the minimal structural unit required to bind with substantial affinity to a particular desired antigen, will therefore typically include the three CDRs, and at least three, preferably four, framework regions interspersed there between to hold and present the CDRs in the appropriate conformation. Classical four chain antibodies have antigen binding sites which are defined by VH and VL domains in cooperation. Certain antibodies, such as camel and shark antibodies, lack light chains and rely on binding sites formed by heavy chains only. Single domain engineered immunoglobulins can be prepared in which the binding sites are formed by heavy chains or light chains alone, in absence of cooperation between VH and VL. In principle, the antigen binding domain may comprise any such polypeptide having the appropriate antigen binding function.

For the avoidance of doubt, unless otherwise indicated, the numbering of residues in the constant domains of an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody. The residues in the V region are numbered according to Kabat numbering unless sequential or other numbering system is specifically indicated.

Spacer Domain The polypeptide of the invention may comprise a spacer sequence to connect the antigen-binding domain with the other domain(s). A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

Production/Expression

Construction and operation of standard expression systems for polypeptide production is well within the ambit of the skilled reader. Such systems are widely commercially available and are used as a matter of routine in order to produce polypeptide molecules having the desired elements such as the CDRs of interest.

A person skilled in the art can make these or any other polypeptide variants according to their choice and/or the desired application. The production of each of these and any other antigen binding domain variants is enabled by the amino acid sequences of the variable regions of the immunoglobulin or antibody of interest, in particular the sequences of the CDRs. For example, in order to produce V_{L} or V_{H} polypeptides, the variable region sequences such as the CDRs (i.e. nucleotide sequence encoding the CDRs or the larger variable regions) may be inserted into a standard heavy/light chain expression vector; more suitably such sequences are joined to nucleotide sequence(s) encoding the other element(s) of the polypeptide of the invention such as the pre-T-alpha domain to directly produce the polypeptide of the invention as a single fusion protein.

Nucleotide Sequences

The invention also relates to nucleic acids and/or nucleotide sequences encoding the polypeptides of the invention. Written description of such nucleotide sequences is effectively provided by disclosure of the amino acid sequences, which by application of the universal genetic code convey the nucleotide sequences to the skilled reader. For completeness, the universal genetic code is provided below:

| | | | |
|---|---|---|---|
| TTT F Phe | TCT S Ser | TAT Y Tyr | TGT C Cys |
| TTC F Phe | TCC S Ser | TAC Y Tyr | TGC C Cys |
| TTA L Leu | TCA S Ser | TAA * Ter | TGA * Ter |
| TTG L Leu | TCG S Ser | TAG * Ter | TGG W Trp |
| CTT L Leu | CCT P Pro | CAT H His | CGT R Arg |
| CTC L Leu | CCC P Pro | CAC H His | CGC R Arg |
| CTA L Leu | CCA P Pro | CAA Q Gln | CGA R Arg |
| CTG L Leu | CCG P Pro | CAG Q Gln | CGG R Arg |
| ATT I Ile | ACT T Thr | AAT N Asn | AGT S Ser |
| ATC I Ile | ACC T Thr | AAC N Asn | AGC S Ser |
| ATA I Ile | ACA T Thr | AAA K Lys | AGA R Arg |
| ATG M Met | ACG T Thr | AAG K Lys | AGG R Arg |
| GTT V Val | GCT A Ala | GAT D Asp | GGT G Gly |
| GTC V Val | GCC A Ala | GAC D Asp | GGC G Gly |
| GTA V Val | GCA A Ala | GAA E Glu | GGA G Gly |
| GTG V Val | GCG A Ala | GAG E Glu | GGG G Gly |

Initiation codon = ATG; termination codons = TAA, TAG, TGA.

It should be noted that nucleotide sequences may be codon optimised, for example codon optimised for humans. Alternatively, or in addition, nucleotide sequences may be codon wobbled. Thus the skilled reader is able to arrive at a nucleotide sequence encoding the polypeptide of interest as a routine matter, and may choose a variant nucleotide sequence (e.g. codon optimised for humans) according to their preferences in operating the invention.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

Inclusion of genetic elements such as origin of replication, promoters for expression of gene(s), selectable markers and the like is well within the ability of the skilled worker manipulating nucleic acids.

The present invention also provides a vector, or kit of vectors which comprises one or more nucleic acid sequence(s) of the invention. Such a vector may be used to introduce the nucleic acid sequence(s) into a host cell so that it expresses the polypeptide(s) of the invention.

The vector may, for example, be a plasmid or a viral vector, such as a retroviral vector or a lentiviral vector, or a transposon based vector or synthetic mRNA.

The vector may be capable of transfecting or transducing a T cell or a NK cell.

Cell

The present invention relates to a cell which comprises a polypeptide according to the present invention.

The cell may comprise a nucleic acid or a vector of the present invention.

The cell may be an immune cell, such as a cytolytic immune cell. Cytolytic immune cells can be T cells or T lymphocytes which are a type of lymphocyte that play a central role in cell-mediated immunity. They can be distinguished from other lymphocytes, such as B cells and natural killer cells (NK cells), by the presence of a T-cell receptor (TCR) on the cell surface.

Complex

Suitably the polypeptide of the invention finds application as part of a complex such as a TCR complex. Suitably said complex comprises one or more further polypeptide(s) as described herein. Suitably said further polypeptide(s) or variant(s) thereof comprise the amino acid sequence of the human polypeptide(s), or comprise the amino acid sequence derived from the human polypeptide(s).

Exemplary sequences for such further polypeptide(s) include:

CD3δ- accession number P04234

CD3ε—accession number P07766

CD3γ—accession number P09693

CD3Z (ζ)—accession number P20963

### Further Applications

The polypeptide, complex, nucleic acid, cell or kit of the invention may be for in vivo or in vitro use. The method is suitably an in vivo or an in vitro method.

Where more than one polypeptide is supplied (for example where costimulatory polypeptides are supplied in trans (such as a CD3E-41BB) fusion; for example when a cognate partner of an activation domain is also supplied (such as FKBP12-CD148 for a polypeptide according to the present invention comprising activation domain FRB) these may be supplied as a nucleic acid comprising a single open reading frame encoding both polypeptides separated by a self-cleaving peptide such as the self-cleaving 2A peptide (SEQ ID NO: 17), or any other suitable self cleaving peptide may be used as appropriate.

Suitably the antigen binding domain and the pre-T-alpha are heterologous. For example suitably the antigen binding domain may be, or be derived from, a non-human animal such as a mouse; suitably the pre-T-alpha is, or is derived from, human.

In another aspect the invention relates to a controllable indirect CAR comprising of an antigen recognition domain fused with a truncated Pre-T-alpha fused with a heterodimerization domain co-expressed with a docking polypeptide comprising a cognate heterodimerization domain and a phosphatase.

In one embodiment suitably the heterodimierization domains assemble in the presence of a small molecule. Suitably the heterodimerization domains are FKBP12, FRB.

In one embodiment suitably the heterodimerization domains dissociate in the presence of a small molecule. Suitably the heterodimerization domains are TetR/Tip.

Pharmaceutical Composition

The present invention also relates to a pharmaceutical composition containing a cell according to the present invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating and/or preventing a disease which comprises the step of administering the cell of the present invention (for example in a pharmaceutical composition as described above) to a subject.

A method for treating a disease relates to the therapeutic use of the cell of the present invention. In this respect, the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample;
(ii) transducing or transfecting such cells with a nucleic acid sequence or vector provided by the present invention;
(iii) administering the cells from (ii) to a subject.

The methods provided by the present invention for treating a disease may involve monitoring the progression of the disease and any toxic activity and administering or removing an agent to inhibit CAR signalling and thereby reduce or lessen any adverse toxic effects.

The methods provided by the present invention for treating a disease may involve monitoring the progression of the disease and monitoring any toxic activity and adjusting the dose of the agent administered to the subject to provide acceptable levels of disease progression and toxic activity.

Monitoring the progression of the disease means to assess the symptoms associated with the disease over time to determine if they are reducing/improving or increasing/worsening.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

### BRIEF DESCRIPTION OF THE DRAWINGS

FIG. 1: Ionizable residues located in the TM domains of the TCRα heterodimer and the CD3δε, CD3γε, and ζζ signaling dimers. (a) Three basic residues are located in the TM domains of the TCR heterodimer, and a pair of acidic residues is located in each of the three signaling dimers. The lysine residues in the TM of TCRα and β are positioned near the center of the predicted TM domains, like the pairs of acidic TM residues in CD3δε and CD3γε. The TM arginine of TCRα and the aspartic acid pair of the ζζ dimer are located in the N-terminal third of the respective TM domains. (b) The three basic TM residues are fully conserved in all TCR forms: the pre-TCR, TCRαβ, and TCRγδ.

FIG. 2: Organization of TCR-CD3 assembly based on the interaction of ionizable TM residues.Site-directedmutagenesisexperimentsdemonstratedthateachbasicTCR Tm residues serve as distinct role in the assembly process and provides an interaction site for one of the three signalling dimers (CD3δε, CD3γε, ζζ). Each assembly step results in the formation of a three-helix interface in the membrane involving one basic TCR TM residue (blue) and a pair of acidic TM residues (red) from the interacting signalling dimer. Formation of the correct receptor structure thus requires proper placement of all nine basic/acidic TM residues.

FIG. 3: Schematic representation of the pTα/TCRβ complex. P represent potential protein kinase phosphorylation sites within the cytoplasmic tail of pTα. Two positively charged amino acids(arginine and lysine) are located in the transmembrane region of pTα. Five potential N-linked glycosylation sites are marked with bars.

FIG. 4: Early stages of development of CD4−CD8− (double negative) and CD4+CD8+(double positive) thymocytes. The early stages of double negative (DN) and double positive (DP) thymocyte development are shown. Levels of pre-T-cell receptor α-chain (Ptcra) mRNA increase up to the DN3 or DN4 stage of development and then decline. Mice that are deficient in the proteins listed show a developmental arrest at the DN3 stage. DNA-PK, DNA-dependent protein kinase; LAT, linker for activation of T cells; RAG, recombination-activating gene; SLP76, SRC-homology-2-domain-containing leukocyte protein of 76 kDa; SYK, spleen tyrosine kinase; TCR, T-cell receptor; ZAP70, ζ-chain-associated protein kinase of 70 kDa.

FIG. 5. (a) Schematic of the CD3/TCR complex; (b) An antigen binding domain (scFv) is attached to the Pre-T-alpha which pairs with the TCR beta chain replacing the TCR alpha chain.

FIG. 6. (a) The endodomain of the chimeric PreTalpha is replaced with the co-stimulatory CD28 endodomain. (b) Additionally, a different type of co-stimulatory domain replaces the CD3 epsilon endodomain.

FIG. 7. (a) The endodomain of the chimeric preTalpha is replaced with TetR. Tip-CD45 endodomain is co-expressed and assembles only in the absence of tetacycline or minocycline. (b) The endodomain of the chimeric preTalpha is splaced with the FRB fragment. This is co-expressed with a fusion protein between FKBP12 and the CD45 endodomain. This assembles only in the presences of Rapamycin or rapalogues.

FIG. 8. Schematic structures of the anti-CD19 pre-TCR alpha constructs and the CD3 complex and TCR beta constructs described in Example 5.

(A-C) The pre-TCR alpha constructs have an N-terminal RQR8 marker followed by a self-cleaving 2A peptide sequence, an anti-CD19 (fmc63) single chain variable fragment (scFv) and a C-terminal pre-TCR alpha chain, either alone (A) or attached to a CD28

(B) or 4-1BB (C) endodomain. (D) The CD3 complex construct is comprised of ζ, γ, δ and ε subunits separated by self-cleaving 2A peptide sequences, followed by an IRES-GFP. (E) The TCR beta construct is comprised of an N-terminal signal sequence followed by a TCR beta chain.

FIG. 9. Anti-CD19 pre-TCR alpha constructs express on the surface of SupT1 cells. (A) SupT1 cells were either non-transduced (NT) or transduced with: the CD3 complex-I-GFP or TCR beta constructs (top panel); the anti-CD19 pre-TCR alpha constructs (middle panel); or triple transduced with the CD3 complex construct, the TCR beta chain and the pre-TCR alpha constructs (bottom panel). (B) Surface expression of the anti-CD19 preTalpha chain was analysed by staining sCD19-Rabbit Fc fusion protein followed by an anti-Rabbit PE. When gated on RQR8+ transduced cells there is detectable surface expression of the pre-Ta constructs (top panel) which was increased when the cells were additionally transduced with the CD3 complex and a TCR beta chain (bottom panel).

FIG. 10. CD69 expression increases on anti-CD19 pre-TCR alpha-expressing SupT1 cells upon co-culturing with CD19-expressing Raji cells. (A-G) SupT1 cells were either untreated, treated with PMA/ionomycin or co-cultured with Raji cells at a 1:4 ratio (SupT1:Raji) for 48 hours, at which point cells were analysed for CD69 expression by flow cytometry. Both SupT1 and Raji cells were included for the CD69 analysis. (B-D) SupT1 cells were gated on RQR8 expression. (E-F) SupT1 cells were gated on RQR8 and GFP expression.

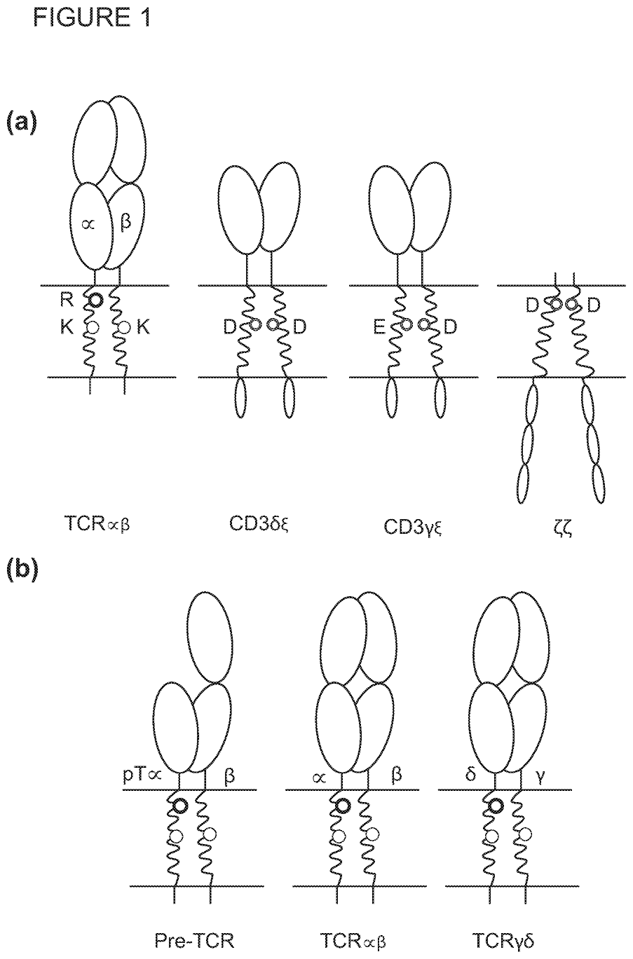

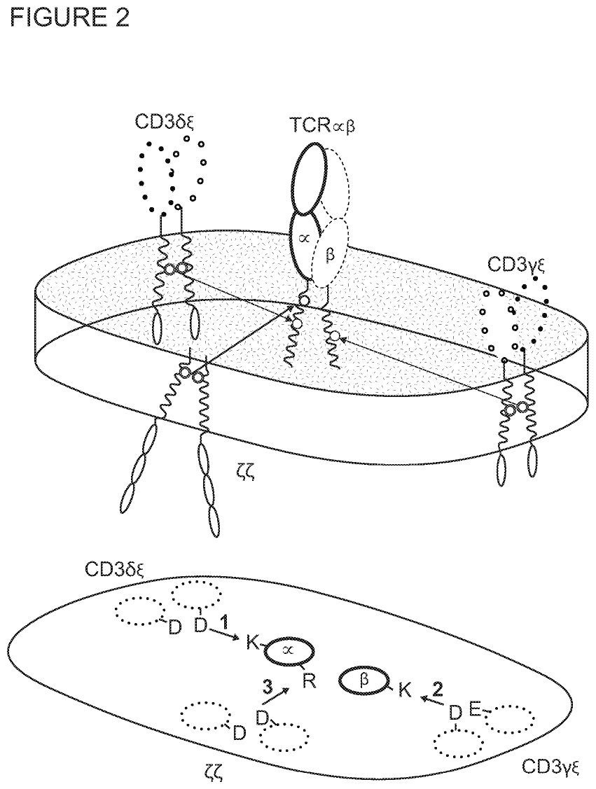

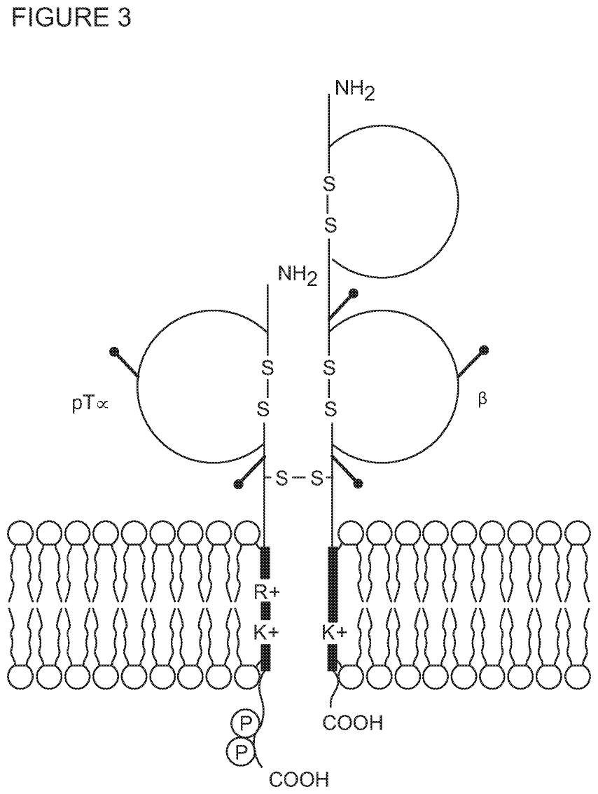

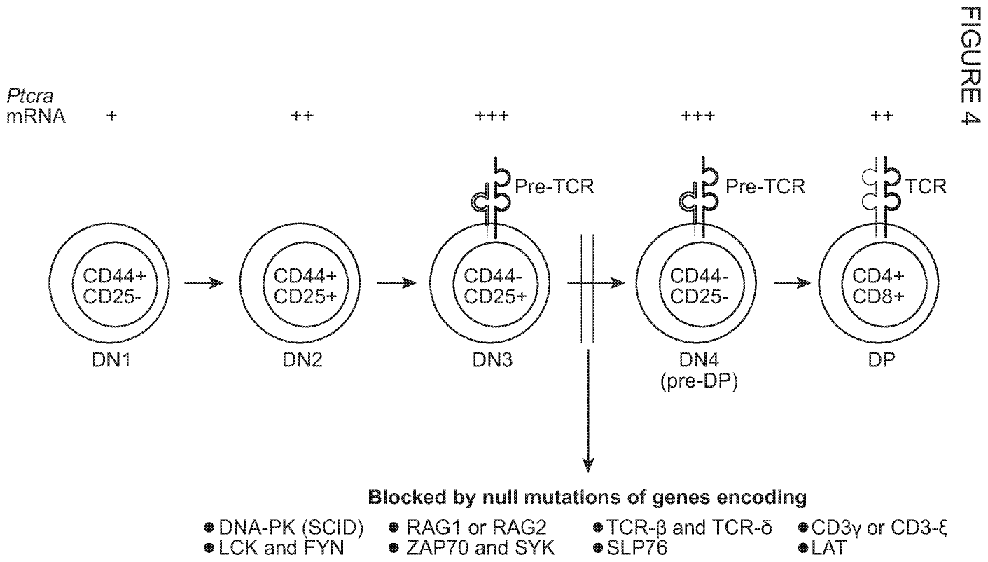

The invention is now described by way of example which is intended to illustrate, rather than limit, embodiments of the invention as set out in the claims.

### EXAMPLES

### Example 1—Production and Functionality of Monolithic Pre-T-Alpha CAR

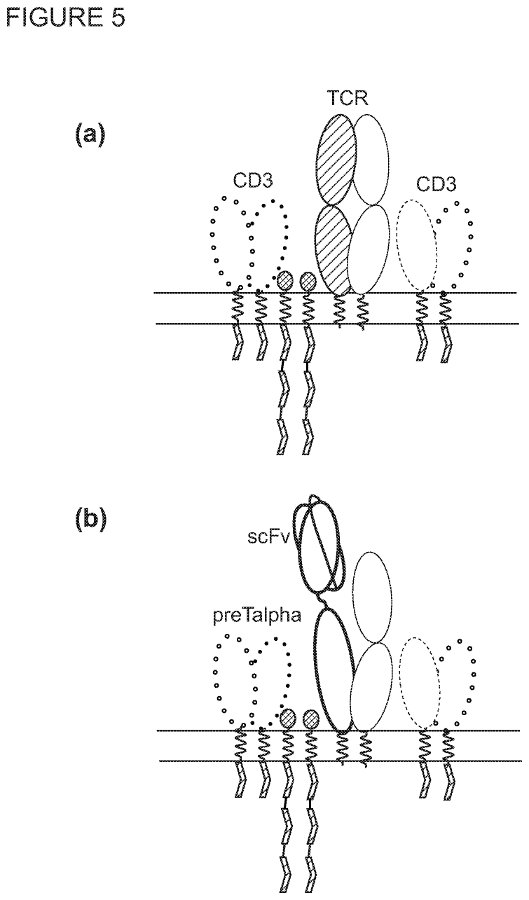

In this example the polypeptide of the invention is a monolithic Pre-T-alpha CAR protein is expressed in the following structure: SFGmR.aCD19_fmc63-dPreTalpha (as illustrated in FIG. 5b)

This comprises of a scFv anti-CD19 (FMC63) attached to the ectodomain and transmembrane from pre-T-alpha chain (PTCRA; Uniprot entry Q6ISU1). Followed by an inert endodomain anchor derived from the first 19 amino acids of human CD19.

Thus in this example the antigen binding domain comprises scFv anti-CD19 (FMC63); the pre-T-alpha domain comprises prises a pre-T-alpha ectodomain (the ectodomain from pre-T-alpha chain (PTCRA; Uniprot entry Q6ISU1)); the antigen binding domain is covalently linked to the pre-T-alpha domain at the N-terminal end of the pre-T-alpha domain because they are joined as a fusion protein. The polypeptide also comprises a transmembrane domain wherein said transmembrane domain is covalently attached to the C-terminal end of the pre-T-alpha domain (again by being placed there in the fusion protein); in this example said transmembrane domain comprises a pre-T-alpha transmembrane domain.

Production of polypeptide according to the invention: the construct is expressed in PBMCs and transduced T-cells are also subjected to in vitro repetitive stimulation and in vivo assays. Primary human T-cells from 3 donors are transduced with four constructs: (i) As a positive control, a “Classical” anti-CD19 CAR; (ii) as another positive control, CD19-TRuC. This is scFv anti-CD19 (FMC63) attached to CD3e as stated in patent WO 2016/187349 A1(TCR2, Inc.); (iii) as a negative control, full length pre-T-alpha and finally (iv) a polypeptide of the invention—the anti-CD19 dPre-T-alpha CAR.

Transduction efficiency is determined using an anti-idiotype to FMC63 followed by flow cytometry.

For in vitro assays; SupT1-GFP cells (which are CD19 negative), are engineered to express CD19 high (>50,000 CD19 molecules per cell) or CD19 low (<1,000 CD19 molecules per cell). Non-transduced T-cells and T-cells transduced with the different CAR constructs are challenged 1:1 with either SupT1 cells or SupT1.CD19 (high or low) cells. Supernatant is sampled 48 or 84 hours after challenge. Supernatant from background (T-cells alone), and maximum (T-cells stimulated with PMA/Ionomycin) are also sampled. During this cytolysis period, the rate of target cell cytolysis is assessed using automated real-time florescence measurements of the target cells. After co-incubation the supernatant is used to measure IFN-γ and IL2 release by ELISA. In addition, the CAR T-cells are collected, counted and assessed for differentiation markers (e.g., CD45RA, CCR7) and exhaustion markers (e.g., PD1, Tim3 or LAG3), TCR downregulation and then re-challenged with new target cells for a further 48 or 84 hours.

The process is repeated with several rounds of stimulation and assessed until there are not enough CAR T-cells to proceed due to a lack of proliferation or T-cell death. Proliferation of T cells and T-cell death is determined using automated real-time fluorescence measurements of the target cells.

In vivo; NSG, female mice aged 6-10 weeks are raised under pathogen free conditions. Mice were sublethally irradiated at 2.8 Gy 1 day prior to intravenous injection with 1×10⁶ F-Luc+ GFP+ NALM6 (CD19+ acute lymphoblastic leukemia). Disease engraftment was assessed by bioluminescent imaging (BLI). Seven days later, 2.5×10⁶ CAR T-cells from the four constructs are administered to NSG. Mice within each cohort are sacrificed at different time-points and engraftment/expansion of T-cells at the tumour bed (bone marrow) or within lymphoid tissues such as lymph nodes, spleen and bone-marrow measured by flow cytometry of said tissues. Mice are used in re-inoculation studies and surviving CAR T cells are used in adoptive cell transfer experiments where CAR T-cells from a cured mouse are transferred into a newly inoculated mouse to assess CAR T cell exhaustion.

### Example 2—Functionality of Co-Stimulatory Domains

The following mono and bicistronic constructs are expressed as a single transcript having the structure:

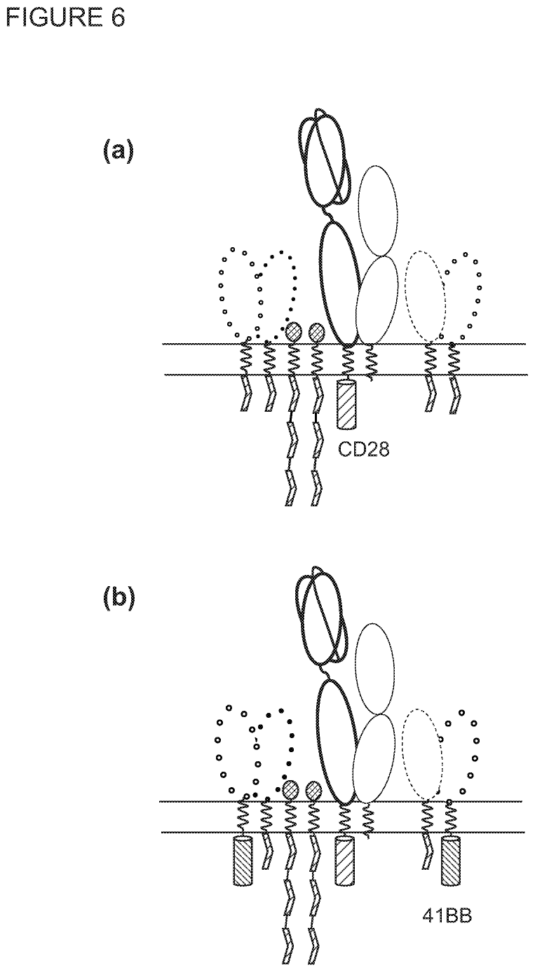

SFGmR.aCD19_fmc63-PreTalpha-CD28 (as illustrated in FIG. 6a) SFGmR.CD3ε-4BB-2A-aCD19_fmc63-PreTalpha-CD28 (as illustrated in FIG. 6b) The monocistronic construct comprises of a scFv anti-CD19 (FMC63) attached to the ectodomain and transmembrane from pre-T-alpha chain (PTCRA; Uniprot entry Q6ISU1). Followed by the endodomain derived from CD28. The bicistronic construct comprises of the ecto and transmembrane domain from human CD3e followed by the endodomain from 41BB (CD137) followed by a self-cleaving 2A peptide, then the scFv anti-CD19 (FMC63) attached to the ectodomain and transmembrane from pre-T-alpha chain (PTCRA; Uniprot entry Q6ISU1). Followed by the endodomain derived from CD28.

The constructs are expressed in PBMCs and transduced T-cells are also subjected to the same in vitro and in vivo assays as stated in Example 1. Primary human T-cells from 3 donors are transduced with three constructs: (i) As a control, the anti-CD19 dPre-T-alpha CAR (ii) the aCD19_fmc63-PreTalpha-CD28 and (iii) CD3ε-41BB-2A-aCD19_fmc63-PreTalpha-CD28. Transduction efficiency is determined using an anti-idotype to FMC63 followed by flow cytometry.

### Example 3—Drug Induced Activation of Pre-T-Alpha CAR

The following bicistronic construct is expressed as a single transcript having the structure:

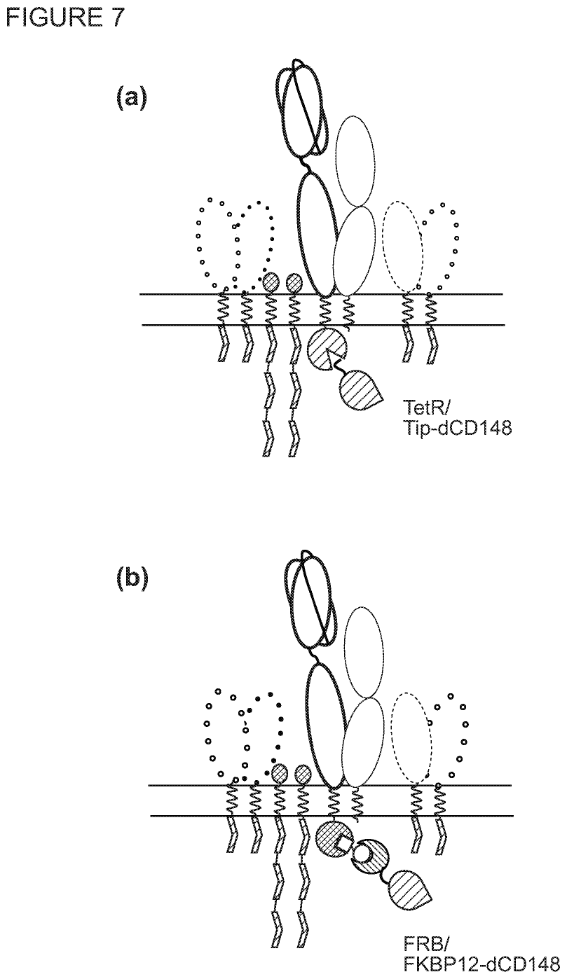

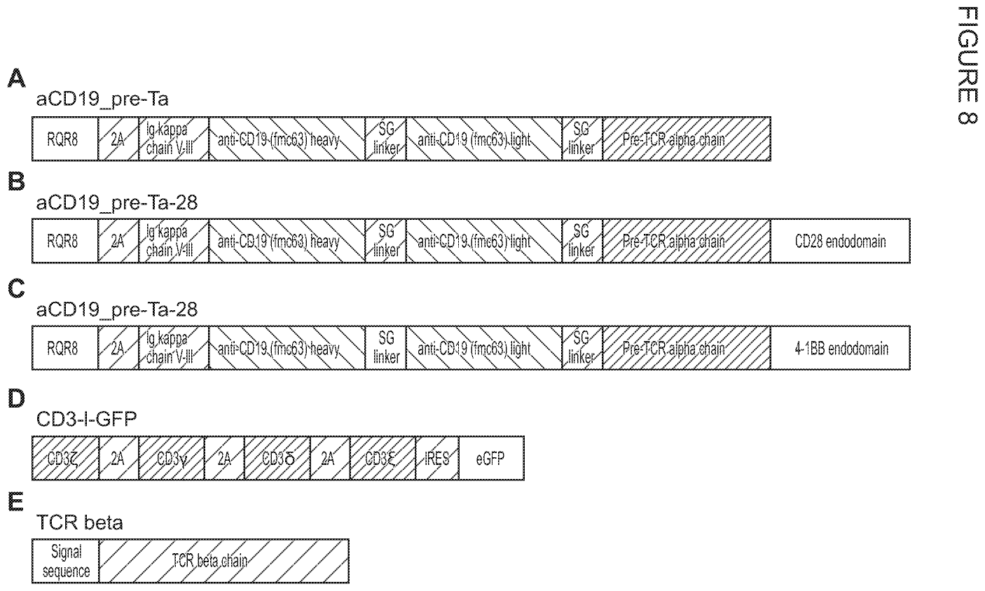

SFGmR.TIP-CD148-2A-fmc63-PreTalpha-TetRB (as illustrated in FIG. 7a)

The bicistronic construct comprises of a TIP peptide (that can compete with tetracycline for binding to the tetRB protein) followed by the endodomain of CD148 (an phosphatases known to inhibit TCR signalling when closely associated with the TCR complex) followed by a self-cleaving 2A peptide, then the scFv anti-CD19 (FMC63) attached to the ectodomain and transmembrane from pre-T-alpha chain (PTCRA; Uniprot entry Q6ISU1). Followed by the TetRB protein.

The construct is expressed in PBMCs and transduced T-cells are also subjected to the same in vitro assays as stated in Example 1 either in the presence or absence of tetracycline.

As illustrated in FIG. 7a, in the absence of tetracycline, the CD48 component dimerises with the membrane tethering component, bringing CD148 into proximity with the intracellular signalling domain of the pre-T-alpha CAR, and dampening cell signalling. In the presence of tetracycline CD148 diffuses freely in the cytoplasm, and the pre-T-alpha CAR-mediated signalling can occur. CAR-mediated activation is therefore “turned up” by the presence of tetracycline.

### Example 4—Drug Induced Inhibition of Pre-T-Alpha CAR

The following bicistronic construct is expressed as a single transcript having the structure:

SFGmR.FKBP12-CD148-2A-fmc63-PreTalpha-FRB (as illustrated in FIG. 7b) The bicistronic construct comprises of FKBP12 domain followed by the endodomain of CD148 (an phosphatases known to inhibit TCR signalling when closely associated with the TCR complex) followed by a self-cleaving 2A peptide, then the scFv anti-CD19 (FMC63) attached to the ectodomain and transmembrane from pre-T-alpha chain (PTCRA; Uniprot entry Q6ISU1). Followed by the FRB domain.

The construct is expressed in PBMCs and transduced T-cells are also subjected to the same in vitro assays as stated in Example 1 either in the presence or absence of rapamycin.

As illustrated in FIG. 7b, in the presence of rapamycin, the CD148 component dimerises with the membrane tethering component, bringing CD148 into proximity with the intracellular signalling domain of the pre-T-alpha CAR, and dampening cell signalling. In the absence of rapamycin CD148 diffuses freely in the cytoplasm, and the pre-T-alpha CAR-mediated signalling can occur. CAR-mediated activation is therefore “turned up” by the presence of rapamycin.

### Example 5—Expression of Pre-T-Alpha CARs in T Cells Leads to T-Cell Activation

Viral vectors comprising each the constructs shown in FIG. 8 were generated and used to transduce T cells. SupT1 cells were either non-transduced (NT); transduced with a single vector expressing:
the CD3 complex-I-GFP shown in FIG. 8 D,
the TCR beta constructs shown in FIG. 8E, or
one of the anti-CD19 pre-TCR alpha construct shown in FIG. 8 A to C;
or triple transduced with vectors expressing (i) the CD3 complex-I-GFP shown in FIG. 8 D, (ii) the TCR beta construct shown in FIG. 8E; and (iii) one of the anti-CD19 pre-TCR alpha constructs shown in FIG. 8 A to C.

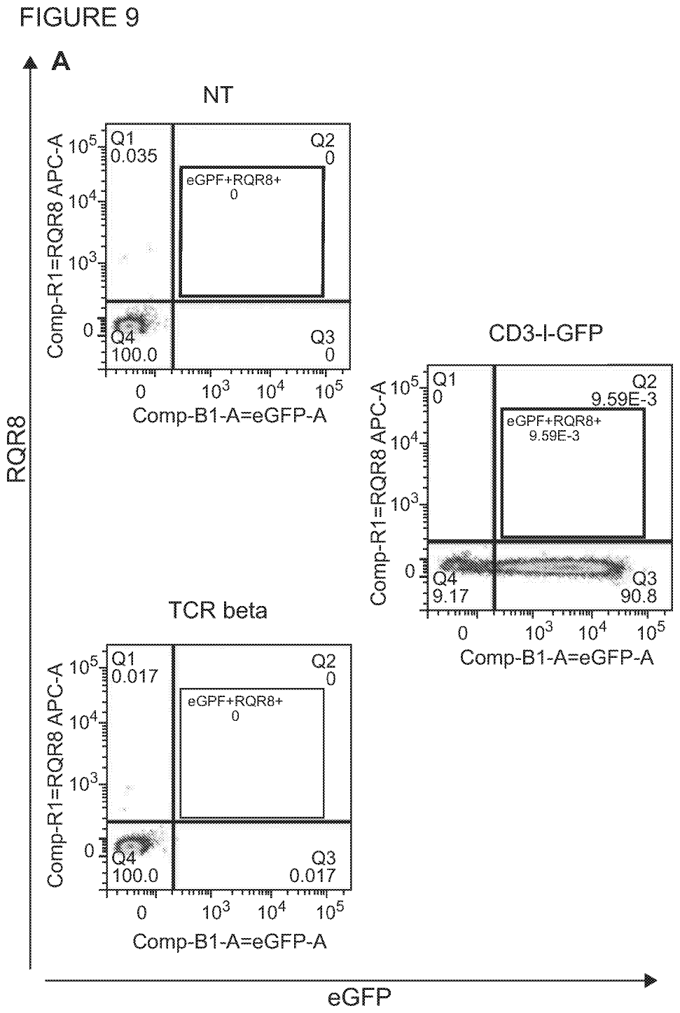

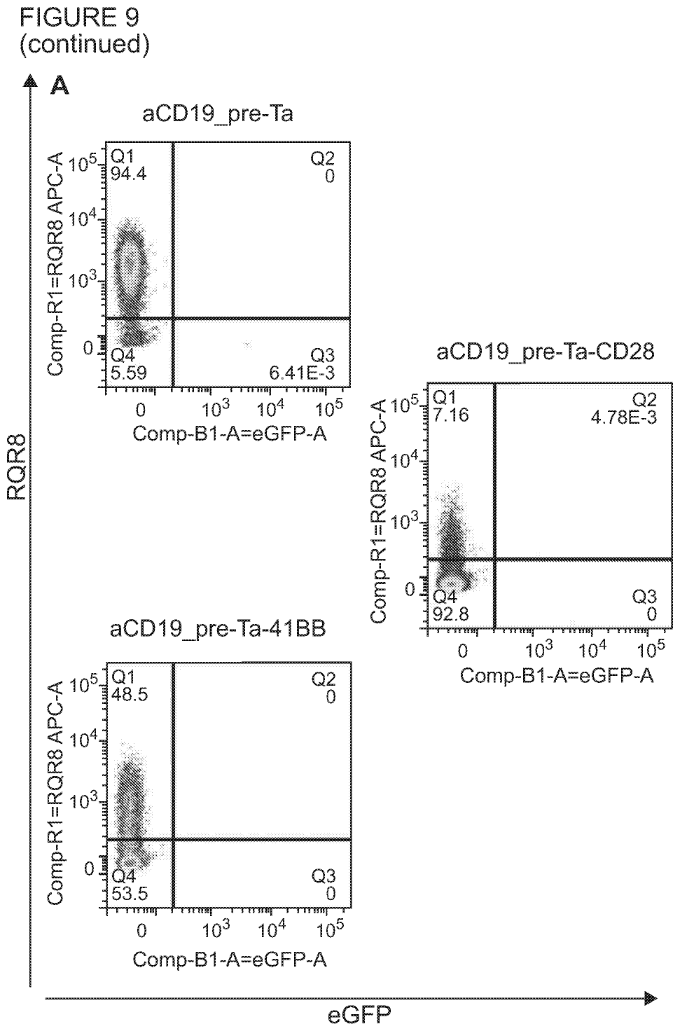

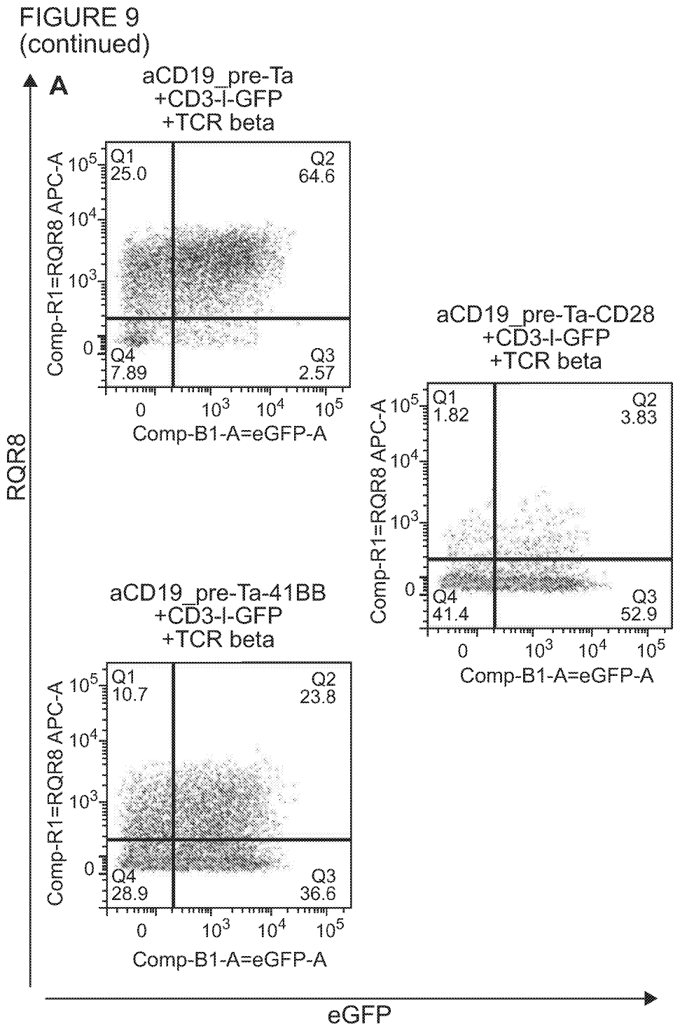

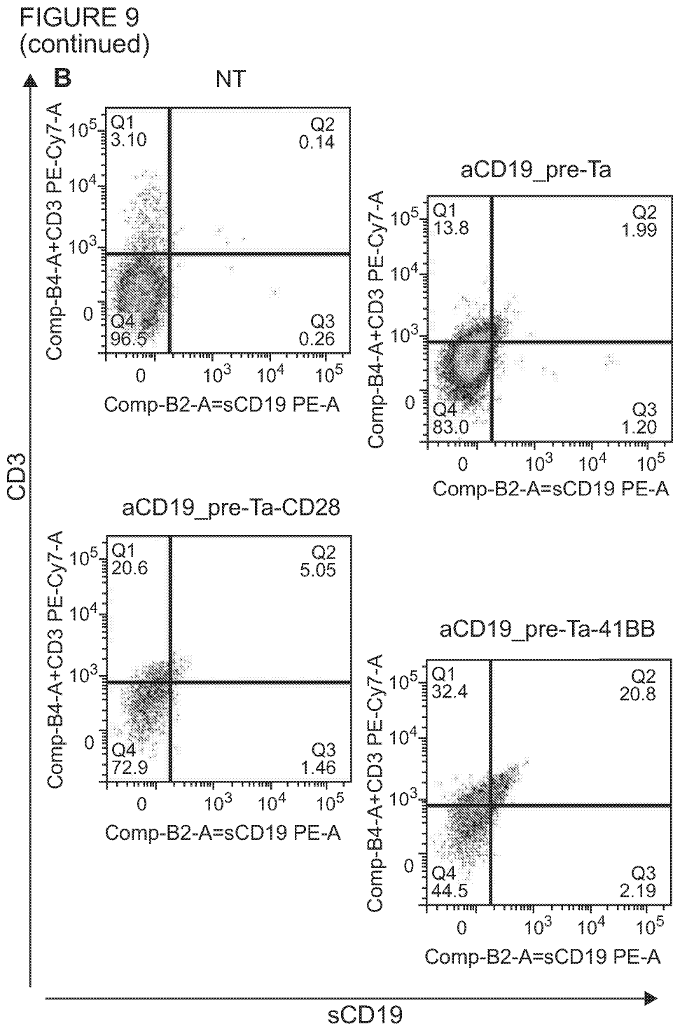

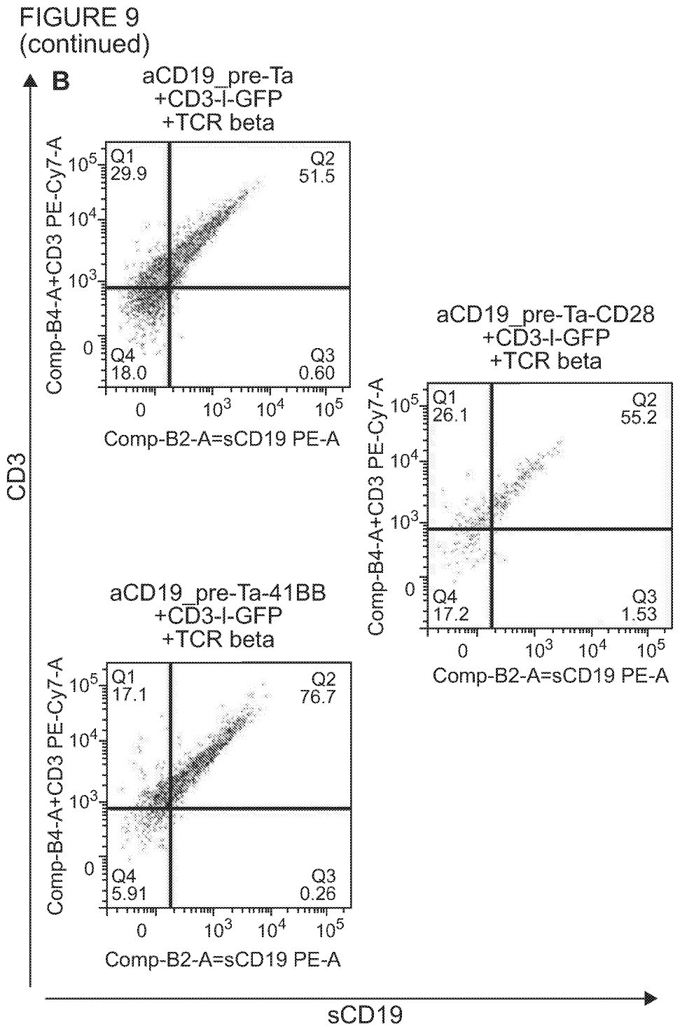

The transductions are shown in FIG. 9A. The triple transduced cells showed improved surface expression levels (bottom panel) compared to cells transduced with vectors expressing the anti-CD19 pre-TCR alpha construct alone (middle panel).

Surface expression of the anti-CD19 preTalpha chain was analysed by staining sCD19-Rabbit Fc fusion protein then labelling with an anti-Rabbit PE and the results are shown in FIG. 9B. Surface expression of the pre-Ta constructs was detectable on cells expressing the RQR8 marker gene (middle panel) which was increased when the cells were additionally transduced with the CD3 complex and a TCR beta chain (bottom panel).

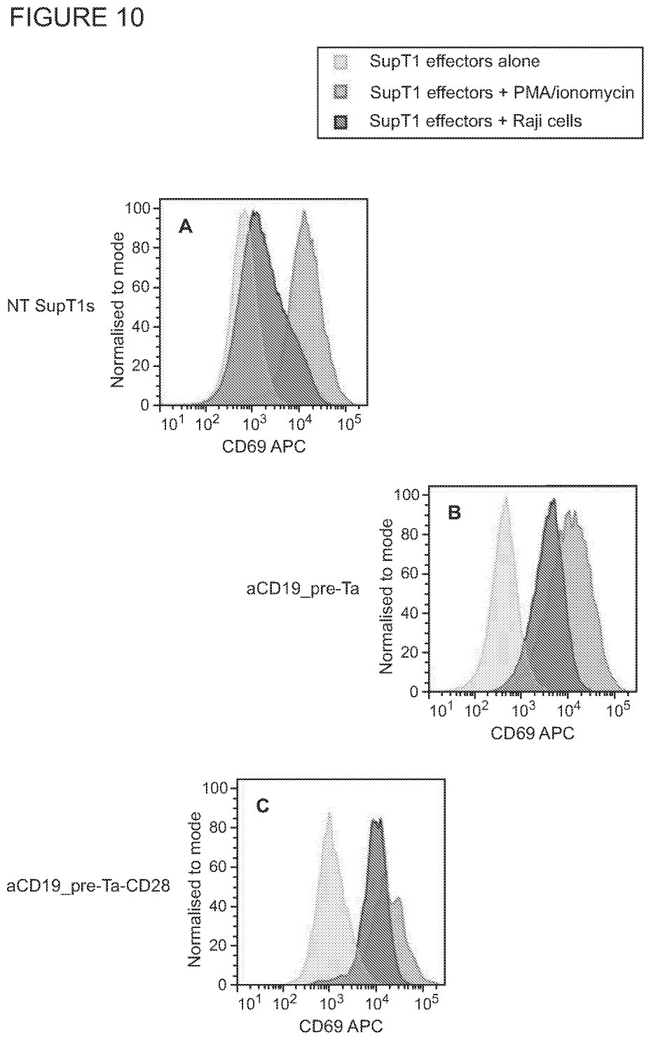

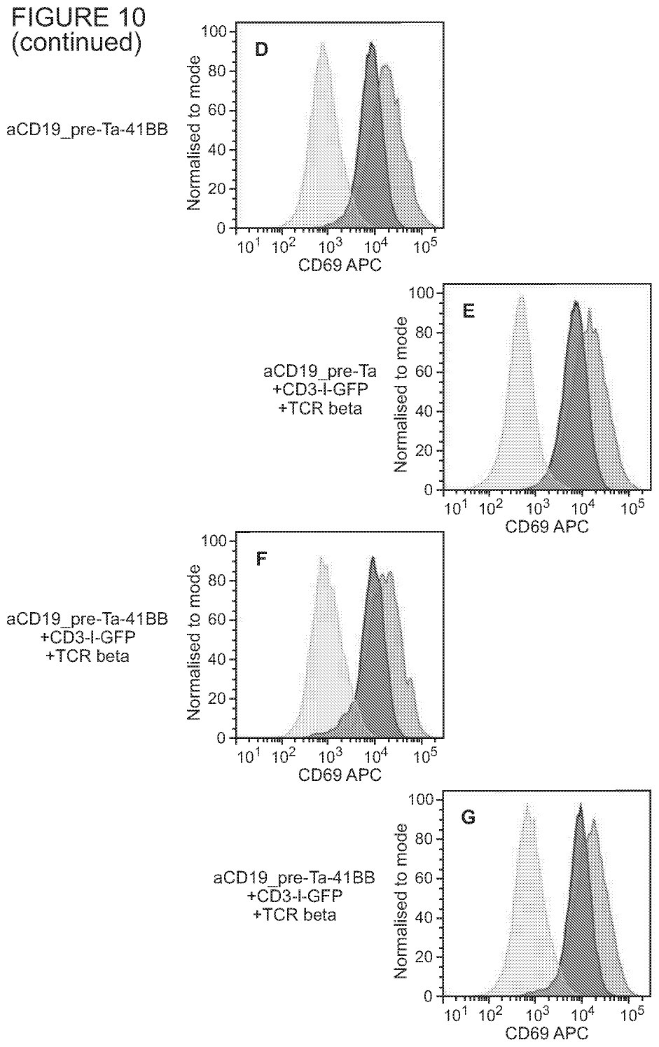

In order to investigate whether expression of anti-CD19 preTalpha causes T cell activation in the presence of CD19+ target cells, the transduced cells described above were either left untreated; treated with PMA/ionomycin; or co-cultured with Raji cells at a 1:4 ratio (SupT1:Raji) for 48 hours, at which point cells were analysed for CD69 expression by flow cytometry. Both SupT1 and Raji cells were included for the CD69 analysis. The results are shown in FIG. 10, in which (B-D) shows cells gated on RQR8 expression and (E-F) shows cells gated on RQR8 and GFP expression. For all anti-CD19 preTalpha constructs, transduced alone or co-transduced with vectors expressing CD3 and TCRbeta, CD69 expression was found to increase on anti-CD19 pre-TCR alpha-expressing SupT1 cells upon co-culturing with CD19-expressing Raji cells. As CD69 is a marker for T-cell activation, this indicates that the expression of anti-CD19 pre-TCR alpha on T cells causes the T cells to become activated in the presence of CD19+ cells.

### REFERENCES

1. L Eyquem, J. et al. Targeting a CAR to the TRAC locus with CRISPR/Cas9 enhances tumour rejection. Nature 543, 113-117 (2017).
2. Gross, G., Gorochov, G., Waks, T. & Eshhar, Z. Generation of effector T cells expressing chimeric T cell receptor with antibody type-specificity. Transplant. Proc. 21, 127-130 (1989).
3. BAEUERLE, P., SIECZKIEWICZ, G. & HOFMEISTER, R. Compositions and Methods for Tcr Reprogramming Using Fusion Proteins. (2017).
4. von Boehmer, H. & Fehling, H. J. Structure and function of the pre-T cell receptor. Annu. Rev. Immunol. 15, 433-452 (1997).
5. Eshhar, Z., Waks, T., Gross, G. & Schindler, D. G. Specific activation and targeting of cytotoxic lymphocytes through chimeric single chains consisting of antibody-binding domains and the gamma or zeta subunits of the immunoglobulin and T-cell receptors. Proc. Natl. Acad. Sci. U.S.A 90, 720-724 (1993).
6. Call, M. E. & Wucherpfennig, K. W. The T cell receptor: critical role of the membrane environment in receptor assembly and function. Annu. Rev. Immunol. 23, 101-125 (2005).
7. Becker, M. L. B. et al. Expression of a hybrid immunoglobulin-T cell receptor protein in transgenic mice. Cell 58, 911-921(1989).
8. Gross, G., Waks, T. & Eshhar, Z. Expression of immunoglobulin-T-cell receptor chimeric molecules as functional receptors with antibody-type specificity. Proc. Natl. Acad. Sci. U.S.A. 86, 10024-10028 (1989).
9. LOEW, A., GRANDA, B. & RAMONES, M. Treatment of Cancer Using Chimeric Cd3 Receptor Proteins. (2017).

Although illustrative embodiments of the invention have been disclosed in detail herein, with reference to the accompanying drawings, it is to be understood that the invention is not limited to the precise embodiment(s) shown and that various changes and modifications can be effected therein by one skilled in the art without departing from the scope of the invention as defined by the appended claims and their equivalents.

All publications mentioned in the above specification are herein incorporated by reference.

1. A polypeptide comprising
(i) an antigen binding domain
(ii) a pre-T-alpha ectodomain; and
(iii) a transmembrane domain.

2-7. (canceled)

8. A polypeptide according to claim 1 further comprising an endodomain.

9. A polypeptide according to claim 8 wherein said endodomain comprises a costimulation domain.

10-12. (canceled)

13. A polypeptide according to claim 9 wherein said costimulation domain comprises one or more of 41BB, OX40, CD27, or TIGR; or CD28 or ICOS costimulation domains.

14. (canceled)

15. A polypeptide according to claim 8 wherein the endodomain comprises, or further comprises, a heterodimerisation domain.

16. A polypeptide according to claim 15 wherein said heterodimerisation domain comprises TetR, Tip, FRB or FKBP12.

17-21. (canceled)

22. A polypeptide according to claim 1 wherein said antigen binding domain comprises a scFv, a dsscFv, a dAb, or a ligand.

23-30. (canceled)

30. A complex comprising a polypeptide according to claim 1, and a TCRβ polypeptide.

31. A complex according to claim 30 wherein said complex further comprises one or more CD3 signalling dimers.

32. A complex according to claim 31 wherein said complex comprises one CD3δε dimer, one CD3γε dimer, and one ζζ dimer.

33. A complex according to claim 30 further comprising a polypeptide comprising a CD3ε-costimulation domain fusion.

34. A complex according to claim 30 wherein when said polypeptide comprises an first heterodimerisation domain, said complex further comprises a docking polypeptide comprising a second heterodimerisation domain which heterodimerises with the first heterodimerisation domain.

35. A nucleotide sequence encoding a polypeptide according to claim 1.

36. A kit comprising
(i) a nucleotide sequence encoding a polypeptide comprising
(a) an antigen binding domain
(b) a pre-T-aloha ectodomain; and
(c) a transmembrane domain
and a first heterodimerisation domain; and
(ii) a nucleotide sequence encoding a docking polypeptide comprising a second heterodimerisation domain which heterodimerises with the first heterodimeerisation domain.

37. A kit comprising
(i) a nucleotide sequence comprising
(a) an antigen binding domain
(b) a pre-T-aloha ectodomain; and
(c) a transmembrane domain, and
(ii) a nucleotide sequence encoding a CD3ε-costimulation domain fusion protein.

38-39. (canceled)

40. A vector comprising a nucleotide sequence according to claim 35.

41. A kit according to claim 36 wherein the nucleotide sequences are vectors.

42. A cell comprising a polypeptide according to claim 1.

43. A method for making a cell according to claim 42, which comprises the step of transducing or transfecting a cell with a nucleic acid encoding a polypeptide comprising
(a) an antigen binding domain
(b) a pre-T-aloha ectodomain; and
(c) a transmembrane domain.

44. A method of treating a subject comprising administering to said subject a cell according to claim 42.

45-47. (canceled)


SEQUENCE LISTING (28 sequences)

SEQ ID NO: 1 - protein, 281 aa, Homo sapiens
MAGTWLLLLLALGCPALPTGVGGTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPVWGEGSYLSSYPTCPAQAWCSRSALRAPSSSLGAFFAGDLPPPLQAGAA

SEQ ID NO: 2 - protein, 23 aa, Homo sapiens
MAGTWLLLLLALGCPALPTGVGG

SEQ ID NO: 3 - protein, 123 aa, Homo sapiens
TPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGG

SEQ ID NO: 4 - protein, 21 aa, Homo sapiens
ALWLGVLRLLLFKLLLFDLLL

SEQ ID NO: 5 - protein, 114 aa, Homo sapiens
TCSCLCDPAGPLPSPATTTRLRALGSHRLHPATETGGREATSSPRPQPRDRRWGDTPPGRKPGSPVWGEGSYLSSYPTCPAQAWCSRSALRAPSSSLGAFFAGDLPPPLQAGAA

SEQ ID NO: 6 - protein, 434 aa, Artificial Sequence
  fmc63-dPreTalpha (figure 5b)
METDTLLLWVLLLWVPGSTGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLQRALVLRRKRKRMTDPTRR

SEQ ID NO: 7 - protein, 20 aa, Artificial Sequence
  fmc63-dPreTalpha (figure 5b)
METDTLLLWVLLLWVPGSTG

SEQ ID NO: 8 - protein, 251 aa, Artificial Sequence
  fmc63-dPreTalpha (figure 5b)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDP

SEQ ID NO: 9 - protein, 19 aa, Artificial Sequence
  Truncated inert endodomain anchor derived from CD19
QRALVLRRKRKRMTDPTRR

SEQ ID NO: 10 - protein, 456 aa, Artificial Sequence
  fmc63-PreTalpha-CD28 (figure 6a)
METDTLLLWVLLLWVPGSTGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLRSKRSRLLHSDYMNMTPRRPGPTRKHYQPYAPPRDFAAYRS

SEQ ID NO: 11 - protein, 41 aa, Artificial Sequence
  Endodomain CD28
RSKRSRLLHSDYMNMTPRRPGPTRKHYQPYAPPRDFAAYRS

SEQ ID NO: 12 - protein, 456 aa, Artificial Sequence; identical to SEQ ID NO: 10
  fmc63-PreTalpha-CD28 (figure 6b)

SEQ ID NO: 13 - protein, 210 aa, Artificial Sequence
  CD3e-41BB-2A (figure 6b)
METDTLLLWVLLLWVPGSTGDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKRGRKKLLYIFKQPFMRPVQTTQEEDGCSCRFPEEEEGGCELEGRGSLLTCGDVEENPGP

SEQ ID NO: 14 - protein, 104 aa, Artificial Sequence
  Ectodomain CD3e
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 15 - protein, 26 aa, Artificial Sequence
  Transmembrane CD3e
VMSVATIVIVDICITGGLLLLVYYWS

SEQ ID NO: 16 - protein, 42 aa, Artificial Sequence
  Endodomain 41BB
KRGRKKLLYIFKQPFMRPVQTTQEEDGCSCRFPEEEEGGCEL

SEQ ID NO: 17 - protein, 18 aa, Artificial Sequence
  2A
EGRGSLLTCGDVEENPGP

SEQ ID NO: 18 - protein, 628 aa, Artificial Sequence
  fmc63-PreTalpha-TetRB (figure 7a)
METDTLLLWVLLLWVPGSTGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLRSKRSRMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 19 - protein, 6 aa, Artificial Sequence
  Linker Anchor
RSKRSR

SEQ ID NO: 20 - protein, 207 aa, Artificial Sequence
  TetRB
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 21 - protein, 376 aa, Artificial Sequence
  TIP-CD148-2A (figure 7a)
MWTWNAYAFAAPSGGGSRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIAEGRGSLLTCGDVEENPGP

SEQ ID NO: 22 - protein, 12 aa, Artificial Sequence
  TIP
MWTWNAYAFAAP

SEQ ID NO: 23 - protein, 5 aa, Artificial Sequence
  Linker
SGGGS

SEQ ID NO: 24 - protein, 341 aa, Artificial Sequence
  Endodomain CD148
RKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 25 - protein, 514 aa, Artificial Sequence
  fmc63-PreTalpha-FRB (figure 7b)
METDTLLLWVLLLWVPGSTGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLRSKRSRILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 26 - protein, 93 aa, Artificial Sequence
  FRB
ILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 27 - protein, 474 aa, Artificial Sequence
  FKBP12-CD148-2A (figure 7b)
MLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGSRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIAEGRGSLLTCGDVEENPGP

SEQ ID NO: 28 - protein, 110 aa, Artificial Sequence
  FKBP12
MLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE